(19) United States
(12) Patent Application Publication
(10) Pub. No.: US 2023/0075570 A1
(43) Pub. Date: Mar. 9, 2023

(54) Methods and Systems for Engineering Power Spectral Features From Biophysical Signals for Use in Characterizing Physiological Systems

(71) Applicant: Analytics for Life Inc., Toronto (CA)

(72) Inventor: Farhad Fathieh, North York (CA)

(21) Appl. No.: 17/891,229

(22) Filed: Aug. 19, 2022

Related U.S. Application Data
(60) Provisional application No. 63/235,963, filed on Aug. 23, 2021.

(51) Int. Cl. A61B 5/021; A61B 5/00; G16H 50/50; G16H 50/30

(52) U.S. Cl. CPC A61B 5/02108; A61B 5/7246; G16H 50/50; G16H 50/30

(57) ABSTRACT

The exemplified methods and systems facilitate the use, for diagnostics, monitoring, treatment, of one or more power spectral-based features or parameters determined from biophysical signals such as cardiac/biopotential signals and/or photoplethysmography signals that are acquired non-invasively from surface sensors placed on a patient while the patient is at rest. The power spectral-based features or parameters can be used in a model or classifier (e.g., a machine-learned classifier) to estimate metrics associated with the physiological state of a patient, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

### RELATED APPLICATION

This US application claims priority to, and the benefit of, U.S. Provisional Patent Application No. 63/235,963, filed Aug. 23, 2021, entitled “Methods and Systems for Engineering Power Spectral Features From Biophysical Signals for Use in Characterizing Physiological Systems,” which is incorporated by reference herein in its entirety.

### FIELD OF THE INVENTIONS

The present disclosure generally relates to methods and systems for engineering features or parameters from biophysical signals for use in diagnostic applications; in particular, the engineering and use of power spectral-based features for use in characterizing one or more physiological systems and their associated functions, activities, and abnormalities. The features or parameters may also be used for monitoring or tracking, controls of medical equipment, or to guide the treatment of a disease, medical condition, or an indication of either.

### BACKGROUND

There are numerous methods and systems for assisting a healthcare professional in diagnosing disease. Some of these involve the use of invasive or minimally invasive techniques, radiation, exercise or stress, or pharmacological agents, sometimes in combination, with their attendant risks and other disadvantages.

Diastolic heart failure, a major cause of morbidity and mortality, is defined as symptoms of heart failure in a patient with preserved left ventricular function. It is characterized by a stiff left ventricle with decreased compliance and impaired relaxation leading to increased end-diastolic pressure in the left ventricle, which is measured through left heart catheterization. Current clinical standard of care for diagnosing pulmonary hypertension (PH), and for pulmonary arterial hypertension (PAH), in particular, involves a cardiac catheterization of the right side of the heart that directly measures the pressure in the pulmonary arteries. Coronary angiography is the current standard of care used to assess coronary arterial disease (CAD) as determined through the coronary lesions described by a treating physician. Non-invasive imaging systems such as magnetic resonance imaging and computed tomography require specialized facilities to acquire images of blood flow and arterial blockages of a patient that are reviewed by radiologists.

It is desirable to have a system that can assist healthcare professionals in the diagnosis of cardiac disease and various other diseases and conditions without the aforementioned disadvantages.

### SUMMARY

A clinical evaluation system and method are disclosed that facilitate the use of one or more power spectral-based features or parameters determined from biophysical signals such as cardiac/biopotential signals and/or photoplethysmography signals that are acquired, in preferred embodiments, non-invasively from surface sensors placed on a patient while the patient is at rest. Power spectral-based features or parameters can include power spectral and cross-spectral (coherence) features or parameters. The power spectral-based features or parameters can be used in a model or classifier (e.g., a machine-learned classifier) to estimate metrics associated with the physiological state of a patient, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

Power spectral analysis (PSA) assesses signal energy (or power) in the frequency domain by decomposing the time-series signals into its frequency components. Cross-spectral power analysis, also referred to as Coherence Spectral Analysis (CSA), assesses the measures of association between the frequency content of two or more time series. Coherence spectral analysis may be performed between two biophysical signals of the same type (e.g., between two channels of photoplethysmographic signals or between two channels of cardiac signals).

The estimation or determined likelihood of the presence or non-presence of a disease, condition, or indication of either can supplant, augment, or replace other evaluation or measurement modalities for the assessment of a disease or medical condition. In some cases, a determination can take the form of a numerical score and related information.

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

As used herein, “metric” refers to an estimation or likelihood of the presence, non-presence, severity, and/or localization (where applicable) of one or more diseases, conditions, or indication(s) of either, in a physiological system or systems. Notably, the exemplified methods and systems can be used in certain embodiments described herein to acquire biophysical signals and/or to otherwise collect data from a patient and to evaluate those signals and/or data in signal processing and classifier operations to evaluate for a disease, condition, or indicator of one that can supplant, augment, or replace other evaluation modalities via one or more metrics. In some cases, a metric can take the form of a numerical score and related information.

In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions. An example indicator of certain forms of heart failure is the presence or non-presence of elevated or abnormal left-ventricular end-diastolic pressure (LVEDP). An example indicator of certain forms of pulmonary hypertension is the presence or non-presence of elevated or abnormal mean pulmonary arterial pressure (mPAP).

### BRIEF DESCRIPTION OF THE DRAWINGS

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a schematic diagram of example modules, or components, configured to non-invasively compute power spectral-based features or parameters to generate one or more metrics associated with the physiological state of a patient in accordance with an illustrative embodiment.

FIG. 2 shows an example biophysical signal capture system or component and its use in non-invasively collecting biophysical signals of a patient in a clinical setting in accordance with an illustrative embodiment.

FIGS. 3A-3B each shows an example method to use power spectral-based features/parameters or their intermediate data in a practical application for diagnostics, treatment, monitoring, or tracking in accordance with an illustrative embodiment.

FIG. 4 illustrates an example power spectral analysis feature computation module configured to determine values of power spectral attributes of an acquired biophysical signal in accordance with an illustrative embodiment.

FIG. 5 illustrates an example coherence analysis feature computation module configured to determine values of cross-power spectral (coherence) attributes between acquired biophysical signals in accordance with an illustrative embodiment.

FIG. 6 illustrates another example power spectral analysis feature computation module configured to determine values of power spectral attributes of an acquired biophysical signal in accordance with an illustrative embodiment.

FIGS. 7A and 7B show example methods of the power spectral analysis feature computation module of FIGS. 4 and 6, respectively, in accordance with an illustrative embodiment.

FIG. 8 shows an example method of the coherence analysis feature computation module of FIG. 5 in accordance with an illustrative embodiment.

FIGS. 9A and 9B show various aspects of the power spectral analysis of the method of FIG. 7A in accordance with an illustrative embodiment.

FIGS. 10A-10D show various aspects of the coherence analysis of the method of FIG. 8 in accordance with an illustrative embodiment.

FIGS. 11A-11E show various aspects of power spectral analysis of the method of FIG. 7B in accordance with an illustrative embodiment.

FIG. 12A shows a schematic diagram of an example clinical and diagnostic system that is configured to use power spectral-based features among other computed features to generate one or more metrics associated with the physiological state of a subject in accordance with an illustrative embodiment.

FIG. 12B shows a schematic diagram of the operation of the example clinical and diagnostic system of FIG. 12A in accordance with an illustrative embodiment.

### DETAILED DESCRIPTION

Each and every feature described herein, and each and every combination of two or more of such features, is included within the scope of the present invention provided that the features included in such a combination are not mutually inconsistent.

While the present disclosure is directed to the practical assessment of biophysical signals, e.g., raw or pre-processed photoplethysmographic signals, biopotential/cardiac signals, etc., in the diagnosis, tracking, and treatment of cardiac-related pathologies and conditions, such assessment can be applied to the diagnosis, tracking, and treatment (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications (e.g., to report the power spectral-based features, parameters, or an intermediate output discussed herein)

The terms “subject” and “patient” as used herein are generally used interchangeably to refer to those who had undergone analysis performed by the exemplary systems and methods.

The term “cardiac signal” as used herein refers to one or more signals directly or indirectly associated with the structure, function, and/or activity of the cardiovascular system—including aspects of that signal's electrical/electrochemical conduction—that, e.g., cause contraction of the myocardium. A cardiac signal may include, in some embodiments, biopotential signals or electrocardiographic signals, e.g., those acquired via an electrocardiogram (ECG), the cardiac and photoplethysmographic waveform or signal capture or recording instrument later described herein, or other modalities.

The term “biophysical signal” as used herein includes but is not limited to one or more cardiac signal(s), neurological signal(s), ballistocardiographic signal(s), and/or photoplethysmographic signal(s), but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “photoplethysmographic signal” as used herein refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation. In some embodiments, ballistocardiographic signals are acquired by wearable devices, such as vibration, acoustic, movement, or orientation-based seismocardiogram (SCG) sensors, which can measure the body's vibrations or orientation as recorded by sensors mounted close to the heart. Seismocardiogram sensors are generally used to acquire “seismocardiogram,” which is used interchangeably with the term “ballistocardiogram” herein. In other embodiments, ballistocardiographic signals may be acquired by external equipment, e.g., bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

In addition, the methods and systems described in the various embodiments herein are not so limited and may be utilized in any context of another physiological system or systems, organs, tissue, cells, etc., of a living body. By way of example only, two biophysical signal types that may be useful in the cardiovascular context include cardiac/biopotential signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography. In another example, the two biophysical signal types can be further augmented by ballistocardiographic techniques.

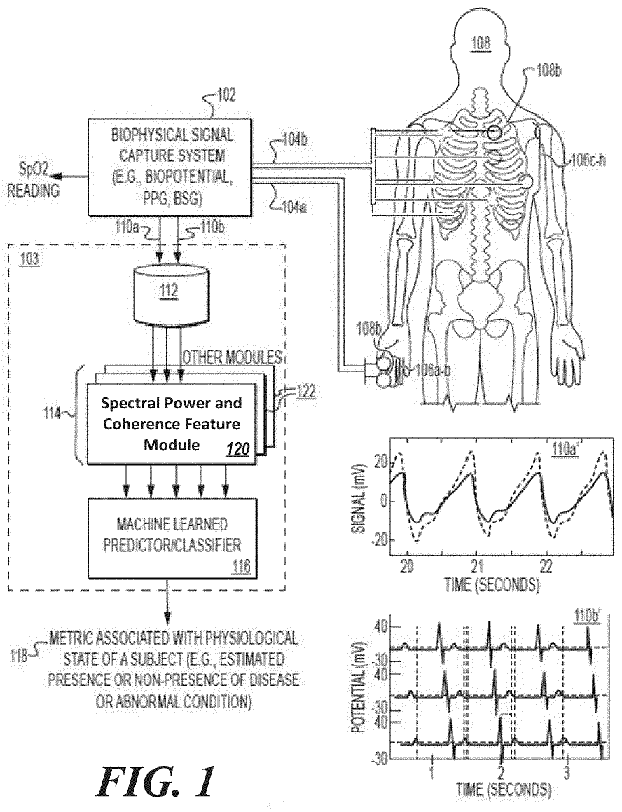

FIG. 1 is a schematic diagram of example modules, or components, configured to non-invasively compute power spectral-based features or parameters to generate, via a classifier (e.g., machine-learned classifier), one or more metrics associated with the physiological state of a patient in accordance with an illustrative embodiment. The modules or components may be used in a production application or the development of the power spectral-based features and other classes of features.

The example analysis and classifiers described herein may be used to assist a healthcare provider in the diagnosis and/or treatment of cardiac- and cardiopulmonary-related pathologies and medical conditions, or an indicator of either. Examples include significant coronary artery disease (CAD), one or more forms of heart failure such as, e.g., heart failure with preserved ejection fraction (HFpEF), congestive heart failure, various forms of arrhythmia, valve failure, various forms of pulmonary hypertension, among various other disease and conditions disclosed herein.

In addition, there exist possible indicators of a disease or condition, such as an elevated or abnormal left ventricular end-diastolic pressure (LVEDP) value as it relates to some forms of heart failure, abnormal left ventricular ejection fraction (LVEF) values as they relate to some forms of heart failure or an elevated mean pulmonary arterial pressure (mPAP) value as it relates to pulmonary hypertension and/or pulmonary arterial hypertension. Indicators of the likelihood that such indicators are abnormal/elevated or normal, such as those provided by the example analysis and classifiers described herein, can help a healthcare provider assess or diagnose that the patient has or does not have a given disease or condition. In addition to these metrics associated with a disease state of condition, other measurements and factors may be employed by a healthcare professional in making a diagnosis, such as the results of a physical examination and/or other tests, the patient's medical history, current medications, etc. The determination of the presence or non-presence of a disease state or medical condition can include the indication (or a metric of measure that is used in the diagnosis) for such disease.

In FIG. 1, the components include at least one non-invasive biophysical signal recorder or capture system 102 and an assessment system 103 that is located, for example, in a cloud or remote infrastructure or in a local system. Biophysical signal capture system 102 (also referred to as a biophysical signal recorder system), in this embodiment, is configured to, e.g., acquire, process, store and transmit synchronously acquired patient's electrical and hemodynamic signals as one or more types of biophysical signals 104. In the example of FIG. 1, the biophysical signal capture system 102 is configured to synchronously capture two types of biophysical signals shown as first biophysical signals 104a (e.g., synchronously acquired to other first biophysical signals) and second biophysical signals 104b (e.g., synchronously acquired to the other biophysical signals) acquired from measurement probes 106 (e.g., shown as probes 106a and 106b, e.g., comprising hemodynamic sensors for hemodynamic signals 104a, and probes 106c-106h comprising leads for electrical/cardiac signals 104b). The probes 106a-h are placed on, e.g., by being adhered to or placed next to, a surface tissue of a patient 108 (shown at patient locations 108a and 108b). The patient is preferably a human patient, but it can be any mammalian patient. The acquired raw biophysical signals (e.g., 106a and 106b) together form a biophysical-signal data set 110 (shown in FIG. 1 as a first biophysical-signal data set 110a and a second biophysical-signal data set 110b, respectively) that may be stored, e.g., as a single file, preferably, that is identifiable by a recording/signal captured number and/or by a patient's name and medical record number.

In the FIG. 1 embodiment, the first biophysical-signal data set 110a comprises a set of raw photoplethysmographic, or hemodynamic, signal(s) associated with measured changes in light absorption of oxygenated and/or deoxygenated hemoglobin from the patient at location 108a, and the second biophysical-signal data set 110b comprises a set of raw cardiac or biopotential signal(s) associated with electrical signals of the heart. Though in FIG. 1, raw photoplethysmographic or hemodynamic signal(s) are shown being acquired at a patient's finger, the signals may be alternatively acquired at the patient's toe, wrist, forehead, earlobe, neck, etc. Similarly, although the cardiac or biopotential signal(s) are shown to be acquired via three sets of orthogonal leads, other lead configurations may be used (e.g., 11 lead configuration, 12 lead configuration, etc.).

Plots 110a′ and 110b′ show examples of the first biophysical-signal data set 110a and the second biophysical-signal data set 110a, respectively. Specifically, Plot 110a′ shows an example of an acquired photoplethysmographic or hemodynamic signal. In Plot 110a′, the photoplethysmographic signal is a time series signal having a signal voltage potential as a function of time as acquired from two light sources (e.g., infrared and red-light source). Plot 110b′ shows an example cardiac signal comprising a 3-channel potential time series plot. In some embodiments, the biophysical signal capture system 102 preferably acquires biophysical signals via non-invasive means or component(s). In alternative embodiments, invasive or minimally-invasively means or component(s) may be used to supplement or as substitutes for the non-invasive means (e.g., implanted pressure sensors, chemical sensors, accelerometers, and the like). In still further alternative embodiments, non-invasive and non-contact probes or sensors capable of collecting biophysical signals may be used to supplement or as substitutes for the non-invasive and/or invasive/minimally invasive means, in any combination (e.g., passive thermometers, scanners, cameras, x-ray, magnetic, or other means of non-contact or contact energy data collection system as discussed herein). Subsequent to signal acquisitions and recording, the biophysical signal capture system 102 then provides, e.g., sending over a wireless or wired communication system and/or a network, the acquired biophysical-signal data set 110 (or a data set derived or processed therefrom, e.g., filtered or pre-processed data) to a data repository 112 (e.g., a cloud-based storage area network) of the assessment system 103. In some embodiments, the acquired biophysical-signal data set 110 is sent directly to the assessment system 103 for analysis or is uploaded to a data repository 112 through a secure clinician's portal.

Biophysical signal capture system 102 is configured with circuitries and computing hardware, software, firmware, middleware, etc., in some embodiments, to acquire, store, transmit, and optionally process both the captured biophysical signals to generate the biophysical-signal data set 110. An example biophysical signal capture system 102 and the acquired biophysical-signal set data 110 are described in U.S. Pat. No. 10,542,898, entitled “Method and Apparatus for Wide-Band Phase Gradient Signal Acquisition,” or U.S. Patent Publication No. 2018/0249960, entitled “Method and Apparatus for Wide-Band Phase Gradient Signal Acquisition,” each of which is hereby incorporated by reference herein in its entirety.

In some embodiments, biophysical signal capture system 102 includes two or more signal acquisition components, including a first signal acquisition component (not shown) to acquire the first biophysical signals (e.g., photoplethysmographic signals) and includes a second signal acquisition component (not shown) to acquire the second biophysical signals (e.g., cardiac signals). In some embodiments, the electrical signals are acquired at a multi-kilohertz rate for a few minutes, e.g., between 1 kHz and 10 kHz. In other embodiments, the electrical signals are acquired between 10 kHz and 100 kHz. The hemodynamic signals may be acquired, e.g., between 100 Hz and 1 kHz.

Biophysical signal capture system 102 may include one or more other signal acquisition components (e.g., sensors such as mechano-acoustic, ballistographic, ballistocardiographic, etc.) for acquiring signals. In other embodiments of the signal capture system 102, a signal acquisition component comprises conventional electrocardiogram (ECG/EKG) equipment (e.g., Holter device, 12 lead ECG, etc.).

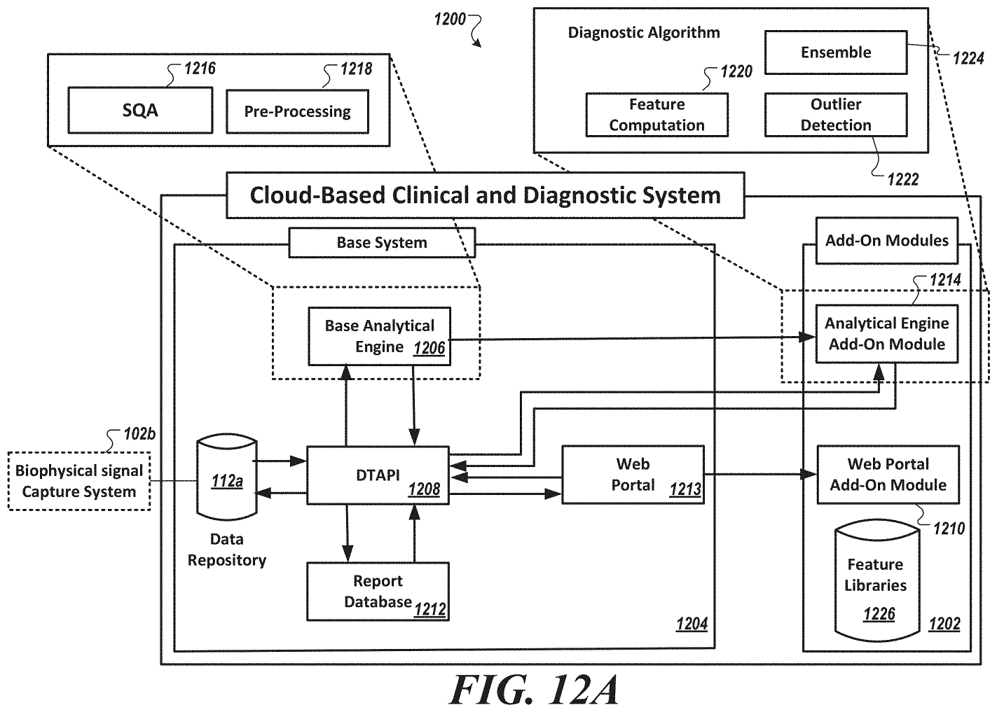

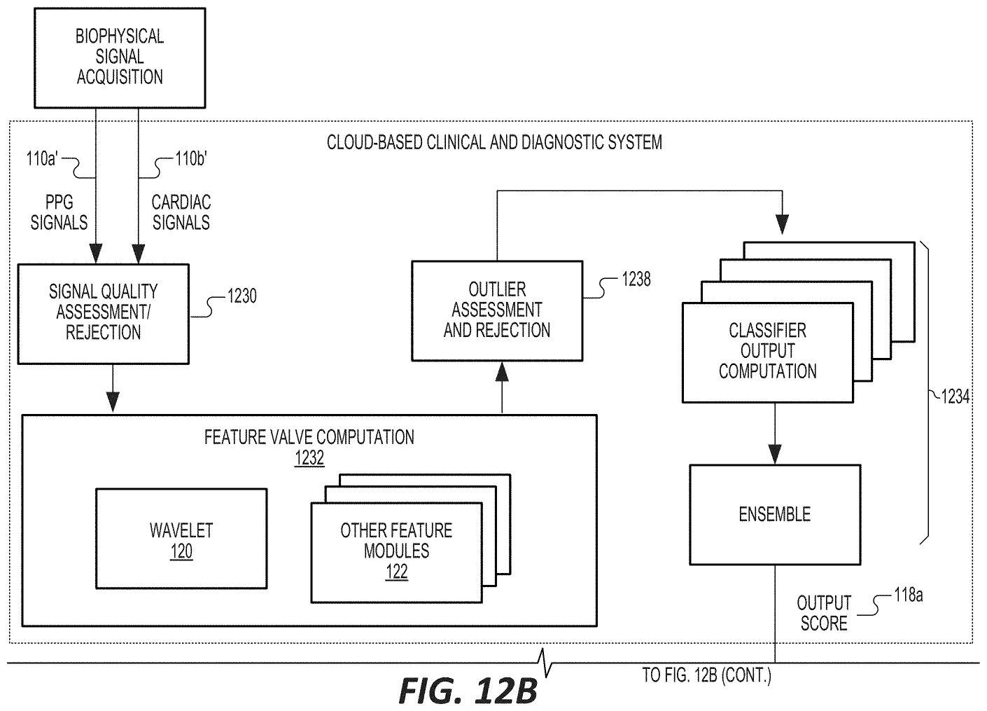

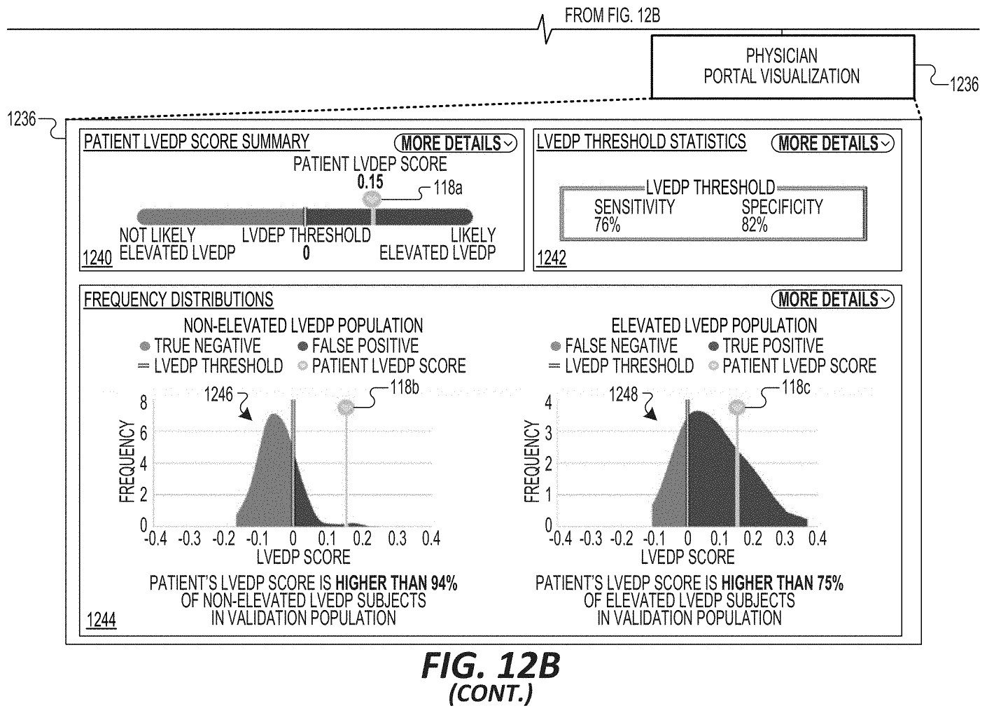

Assessment system 103 comprises, in some embodiments, the data repository 112 and an analytical engine or analyzer (not shown—see FIGS. 12A and 12B). Assessment system 103 may include feature modules 114 and a classifier module 116 (e.g., an ML classifier module). In FIG. 1, Assessment system 103 is configured to retrieve the acquired biophysical signal data set 110, e.g., from the data repository 112, and use it in the feature modules 114, which is shown in FIG. 1 to include a power spectral-feature module 120 and other modules 122 (later described herein). The features modules 114 compute values of features or parameters, including those of power spectral-based features, to provide to the classifier module 116, which computes an output 118, e.g., an output score, of the metrics associated with the physiological state of a patient (e.g., an indication of the presence or non-presence of a disease state, medical condition, or an indication of either). Output 118 is subsequently presented, in some embodiments, at a healthcare physician portal (not shown—see FIGS. 12A and 12B) to be used by healthcare professionals for the diagnosis and treatment of pathology or a medical condition. In some embodiments, a portal may be configured (e.g., tailored) for access by, e.g., patients, caregivers, researchers, etc., with output 118 configured for the portal's intended audience. Other data and information may also be a part of output 118 (e.g., the acquired biophysical signals or other patient's information and medical history).

Classifier module 116 (e.g., ML classifier module) may include transfer functions, look-up tables, models, or operators developed based on algorithms such as but not limited to decision trees, random forests, neural networks, linear models, Gaussian processes, nearest neighbor, SVMs, Naïve Bayes, etc. In some embodiments, classifier module 116 may include models that are developed based on ML techniques described in U.S. Provisional Patent Application No. 63/235,960, filed Aug. 23, 2021, entitled “Method and System to Non-Invasively Assess Elevated Left Ventricular End-Diastolic Pressure” having attorney docket no. 10321-048pv1; U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

Example Biophysical Signal Acquisition.

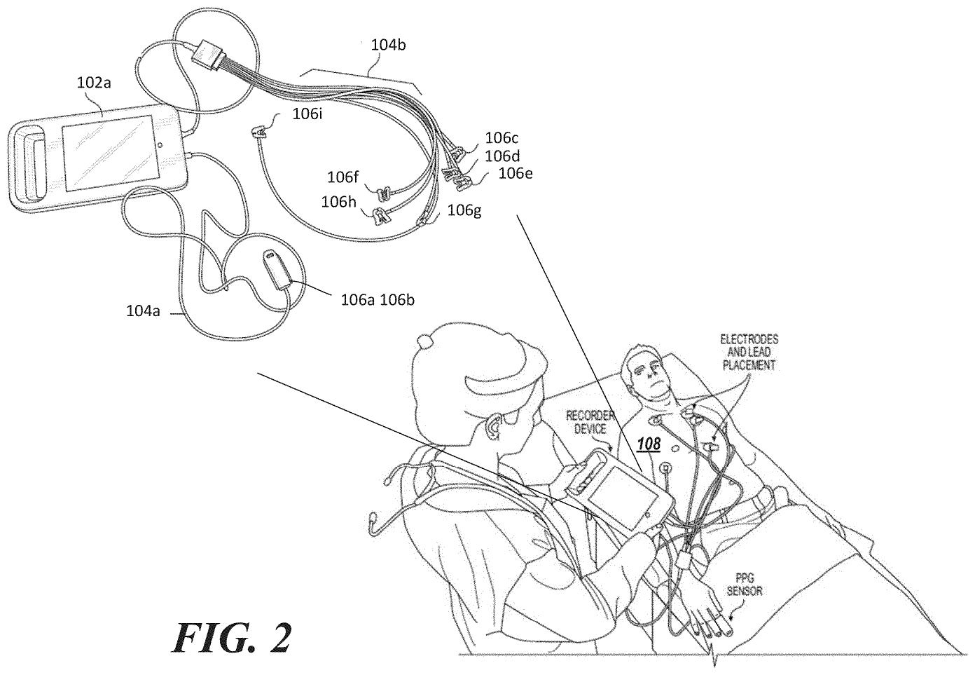

FIG. 2 shows a biophysical signal capture system 102 (shown as 102a) and its use in non-invasively collecting biophysical signals of a patient in a clinical setting in accordance with an illustrative embodiment. In FIG. 2, the biophysical signal capture system 102a is configured to capture two types of biophysical signals from the patient 108 while the patient is at rest. The biophysical signal capture system 102a synchronously acquires the patient's (i) electrical signals (e.g., cardiac signals corresponding to the second biophysical-signal data set 110b) from the torso using orthogonally placed sensors (106c-106h; 106i is a 7^{th} common-mode reference lead) and (ii) hemodynamic signals (e.g., PPG signals corresponding to the first biophysical-signal data set 110a) from the finger using a photoplethysmographic sensor (e.g., collecting signals 106a, 106b).

As shown in FIG. 2, the electrical and hemodynamic signals (e.g., 104a, 104b) are passively collected via commercially available sensors applied to the patient's skin. The signals may be acquired beneficially without patient exposure to ionizing radiation or radiological contrast agents and without patient exercise or the use of pharmacologic stressors. The biophysical signal capture system 102a can be used in any setting conducive for a healthcare professional, such as a technician or nurse, to acquire the requisite data and where a cellular signal or Wi-Fi connection can be established.

The electrical signals (e.g., corresponding to the second biophysical signal data set 110b) are collected using three orthogonally paired surface electrodes arranged across the patient's chest and back along with a reference lead. The electrical signals are acquired, in some embodiments, using a low-pass anti-aliasing filter (e.g., ˜2 kHz) at a multi-kilohertz rate (e.g., 8 thousand samples per second for each of the six channels) for a few minutes (e.g., 215 seconds). In alternative embodiments, the biophysical signals may be continuously/intermittently acquired for monitoring, and portions of the acquired signals are used for analysis. The hemodynamic signals (e.g., corresponding to the first biophysical signal data set 110a) are collected using a photoplethysmographic sensor placed on a finger. The photo-absorption of red light (e.g., any wavelengths between 600-750 nm) and infrared light (e.g., any wavelengths between 850-950 nm) are recorded, in some embodiments, at a rate of 500 samples per second over the same period. The biophysical signal capture system 102a may include a common mode drive that reduces common-mode environmental noise in the signal. The photoplethysmographic and cardiac signals were simultaneously acquired for each patient. Jitter (inter-modality jitter) in the data may be less than about 10 microseconds (μs). Jitter among the cardiac signal channels may be less than 10 microseconds, e.g., around ten femtoseconds (fs).

A signal data package containing the patient metadata and signal data may be compiled at the completion of the signal acquisition procedure. This data package may be encrypted before the biophysical signal capture system 102a transfers the package to the data repository 112. In some embodiments, the data package is transferred to the assessment system (e.g., 103). The transfer is initiated, in some embodiments, following the completion of the signal acquisition procedure without any user intervention. The data repository 112 is hosted, in some embodiments, on a cloud storage service that can provide secure, redundant, cloud-based storage for the patient's data packages, e.g., Amazon Simple Storage Service (i.e., “Amazon S3”). The biophysical signal capture system 102a also provides an interface for the practitioner to receive notification of an improper signal acquisition to alert the practitioner to immediately acquire additional data from the patient.

Example Method of Operation

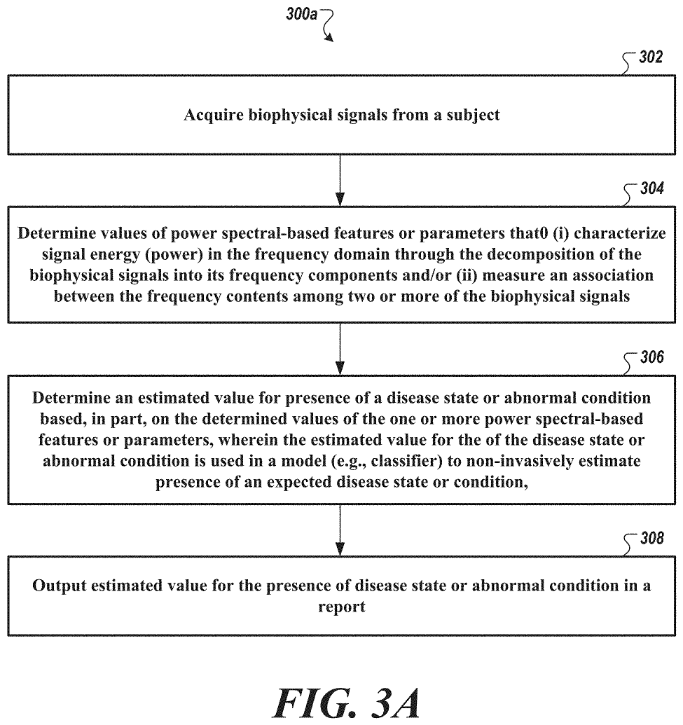

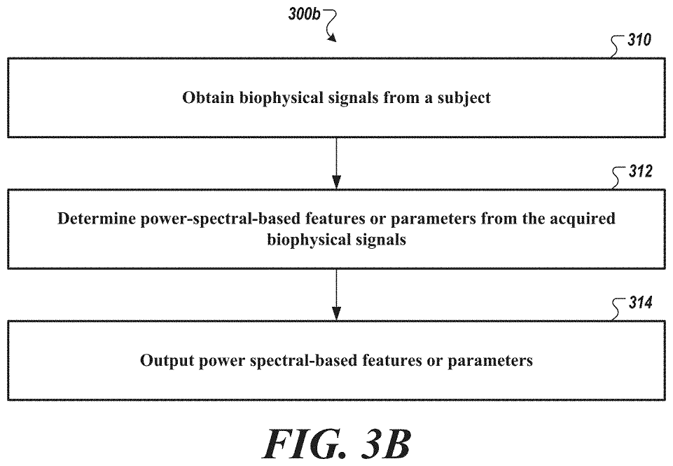

FIGS. 3A-3B each shows an example method to use power spectral-based features/parameters or their intermediate outputs in a practical application for diagnostics, treatment, monitoring, or tracking.

Estimation of Presence of Disease State or Indicating Condition. FIG. 3A shows a method 300a that employs power spectral-based parameters or features to determine estimators of the presence of a disease state, medical condition, or indication of either, e.g., to aid in the diagnosis, tracking, or treatment. Method 300a includes the step of acquiring (302) biophysical signals from a patient (e.g., cardiac signals, photoplethysmographic signals, ballistocardiographic signals), e.g., as described in relation to FIGS. 1 and 2 and other examples as described herein. In some embodiments, the acquired biophysical signals are transmitted for remote storage and analysis. In other embodiments, the acquired biophysical signals are stored and analyzed locally.

As stated above, one example in the cardiac context is the estimation of the presence of abnormal left-ventricular end-diastolic pressure (LVEDP) or mean pulmonary artery pressure (mPAP), significant coronary artery disease (CAD), abnormal left ventricular ejection fraction (LVEF), and one or more forms of pulmonary hypertension (PH), such as pulmonary arterial hypertension (PAH). Other pathologies or indicating conditions that may be estimated include, e.g., one or more forms of heart failure such as, e.g., heart failure with preserved ejection fraction (HFpEF), arrhythmia, congestive heart failure, valve failure, among various other diseases and medical conditions disclosed herein.

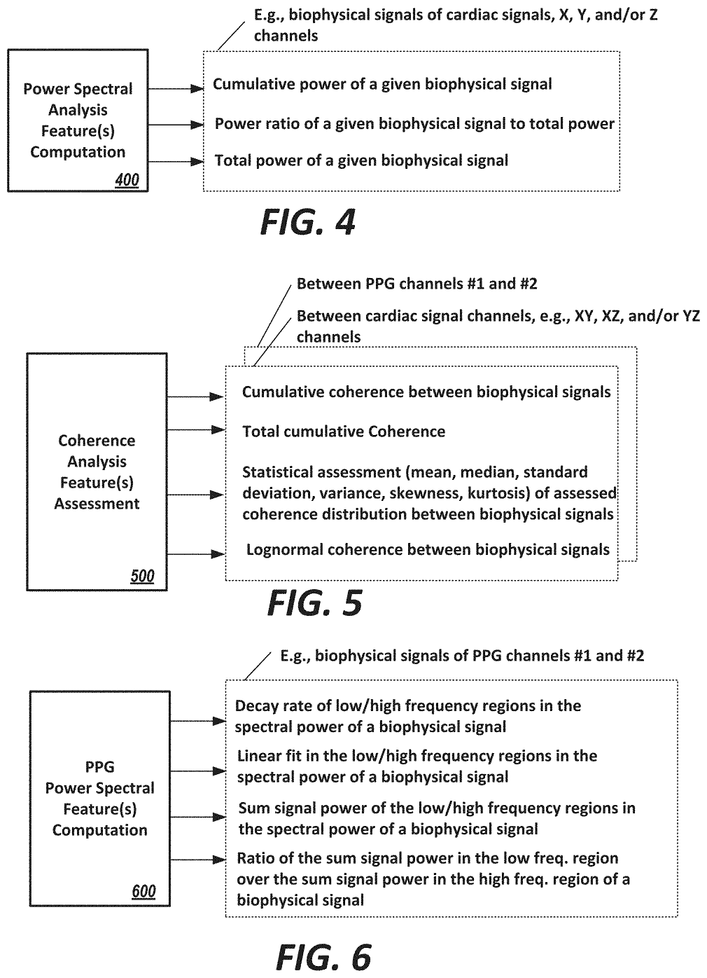

Method 300a further includes the step of retrieving (304) the data set and determining values of power spectral-based features or parameters that i) characterize signal energy (power) in the frequency domain through the decomposition of the two or more biophysical signals into its frequency components and/or ii) measure an association between the frequency content of the two or more biophysical signals. Example operations to determine the values of power spectral-based features or parameters are provided in relation to FIGS. 4-12 later discussed herein. Method 300a further includes the step of determining (306) an estimated value for a presence of a disease state, medical condition, or an indication of either based on an application of the determined power spectral-based features to an estimation model (e.g., ML models). An example implementation is provided in relation to FIGS. 12A and 12B.

Method 300a further includes the step of outputting (308) estimated value(s) for the presence of disease state or abnormal condition in a report (e.g., to be used diagnosis or treatment of the disease state, medical condition, or indication of either), e.g., as described in relation to FIGS. 1, 12A, and 12B and other examples described herein.

Diagnostics or Condition Monitoring or Tracking using Power Spectral-based Features or Parameters. FIG. 3B shows a method 300b that employs power spectral-based features or parameters or features for monitoring or controls of medical equipment or health monitoring device. Method 300b includes the step of obtaining (302) biophysical signals from a patient (e.g., cardiac signals, photoplethysmographic signals, ballistocardiographic signals, etc.). The operation may be performed continuously or intermittently, e.g., to provide output for a report or as controls for the medical equipment or the health monitoring device.

Method 300b further includes determining (310) power spectral-based features or parameters from the acquired biophysical data set, e.g., as described in relation to FIGS. 4-11. The determination based may be based on an analysis of the continuously acquired signals over a moving window.

Method 300b further includes outputting (312) power spectral-based features or parameters (e.g., in a report for use in diagnostics or as signals for controls). For monitoring and tracking, the power spectral-based features or parameters can provide a characterization of signal energy (power) in the frequency domain and/or measure an association between the frequency content of the two or more biophysical signals. The output may be via a wearable device, a handheld device, or medical diagnostic equipment (e.g., pulse oximeter system, wearable health monitoring systems). In some embodiments, the output may be via a point of care monitoring, such as a mobile cart or cart trolley. In some embodiments, the outputs may be used in resuscitation systems, cardiac or pulmonary stress test equipment, pacemakers, etc., in which frequency spectral information is desired.

Spectral Power-Based Features or Parameters

Studies have been performed to investigate the frequency that constitutes biological signals (e.g., cardiac and brain electrical activities) and to assess their diagnostic efficacy. The exemplary system and method employ engineered power spectral-based features or parameters to assess the information embedded in the constituent frequency of cardiac signals and photoplethysmographic signals, among other biophysical signals, in predicting or estimating a metric associated with a disease state or abnormal health condition.

It has been reported that ECG representation in the frequency domain can be employed for human identification with a high identification rate [1]. Another study proposed a heartbeat discrimination method based on ECG frequency-domain feature, extracted from Fourier spectrum of signal ranging from 0 Hz to 20 Hz, that can effectively classify heartbeat classes such as normal beat, supraventricular ectopic beat, bundle branch ectopic beat, and cardiac arrhythmias [2], [3]. Fourier spectrum has also been found to be effective for the classification of ventricular depolarization waves with different morphologies [4]. A machine-based classification algorithm was developed to discriminate the cardiac activity in different states (e.g., resting, fear, exercising, and smoking) [5]. The input features to the classifier were extracted through linear discriminative analysis and cross-correlation of the Fast Fourier Transform (FFT) spectrum of cardiac signals with predefined classes of ECG signals. Besides the cardiac beat classification, frequency-domain features have been reported to discriminate between healthy and subjects with a condition of interest. Analysis of the frequency content of low heart rate variability (HRV) revealed that HRV is associated with an increased risk of sudden cardiac death in the general population [6]. Analyzing the distribution of signal power in the frequency domain (power spectral density), the values of power and frequency of the peaks within specified frequency sub-bands to classify three cardiac conditions: healthy, arrhythmic, and ischemic [7]. It has also been reported that the high frequency and very low-frequency content of HRV have an association with depressive symptoms in children and adolescents [8]. Power spectral analysis of cardiac signals has also been proved to identify obstructive sleep apnea-hypopnea epochs [9]. Moreover, combined multi-channel FFT coefficients with a multivariate linear regression have been shown to have diagnostic capabilities for pulmonary hypertension [10]. High frequency spectral analysis of QRS complex has also been employed to detect IHD and CAD where it revealed significant difference in IHD and CAD positive versus healthy groups. [17-18].

Power Spectral analysis and coherence analysis, as disclosed herein, can be beneficially used in conjunction with acquisition measurements of surface sensors placed on the body (and machine learning algorithms) to estimate the presence, non-presence, severity, and/or localization of elevated or abnormal left-ventricular end-diastolic pressure (LVEDP), significant coronary artery disease (CAD), pulmonary hypertension (PH) or pulmonary arterial hypertension (PAH), abnormal left ventricular ejection fraction (LVEF), heart failure with preserved ejection fraction (HFpEF), or other disease and conditions discussed herein. The frequency representation of a time series is achievable using mathematical transformation, such as Fourier Transform (FT), which preserves all the time-domain information [11]. This is a useful property of such transformations that allows for periodic and quasi-periodic patterns to become more apparent in the frequency domain that may not reveal themselves in the time domain.

Power Spectral and Cross-Power Spectral (Coherence) Features or Parameters

FIGS. 4, 5, and 6 each shows an example power spectral analysis and coherence analysis feature computation module, for a total of three example modules, configured to determine values of power spectral and cross-power spectral (coherence) features or parameters of biophysical signals in accordance with an illustrative embodiment. The power spectral analysis feature computation modules 400 and 600 of FIGS. 4 and 6 each determines the spectral power attributes, as power spectral and coherence-based features or parameters, of cardiac/biopotential signals and photoplethysmographic signals, respectively, in the frequency domain using power spectral analysis operations. Both Modules 400 and 600 can calculate the spectral power of a cardiac signal or a photoplethysmographic signal, respectively, though they may also perform similar computation for other biophysical signals. Module 600 performs further computations that assess the low- and high-frequency portions of the frequency spectrum. The coherence feature computation module 500 of FIG. 5 determines the cross-spectral power attributes between biophysical signals (e.g., between cardiac/biopotential signals or between photoplethysmographic signals) in the frequency domain using coherence analysis operations.

### Example #1—Power Spectral Features or Parameters

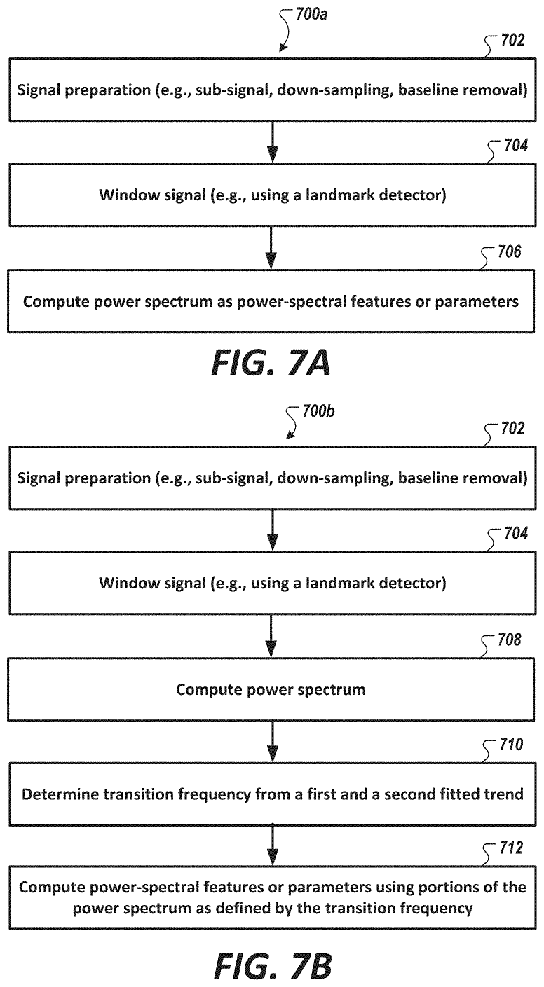

FIG. 4 illustrates, as the first of three example feature or parameter categories, an example power spectral analysis feature computation module 400 configured to determine the spectral power attributes of a set of cardiac signals, including the cumulative spectral power for a given channel, the total spectral power of these channels, and their ratios. FIG. 7A discussed below shows an example method of the power spectral analysis feature computation module of FIG. 4.

Table 1 shows an example set of 3 types of extractable power spectral features and their corresponding description to provide up to 7 features or parameters.

TABLE 1

| Feature name | Description |
|---|---|
| orth_power | Cumulative spectral power (e.g., Pₓ, P_{y}, P_{z}) in |
| | a biophysical signal (e.g., in channels X, Y, or |
| | Z in a cardiac signal) |
| total_power | Sum of cumulative spectral power in the |
| | biophysical signals, channels (e.g., P_{X+Y+Z} = |
| | P_{X} + P_{Y} + P_{Z}) |
| orth_powerRatio | Relative spectral power of a channel to the |
| | total power; Power ratio: Pₙ/P_{X+Y+Z} |

FIG. 7A shows a method 700a to generate power spectral-based features or parameters, e.g., as performed by the power spectral analysis feature computation module 400 of FIG. 4, in accordance with an illustrative embodiment, which can be used wholly, or partially, to generate power spectral-based features or parameters and its outputs to be used in machine-learned classifier to determine a metric associated with a physiological system of a subject under study. To determine the features of Table 1, Module 400 is configured to (i) pre-process (702) the acquired biophysical signal, (ii) window (704) the signals, and (iii) determine (706) power spectrum of the windowed signals as the power spectral features or parameters.

Pre-processing. Module 400 may perform pre-processing (702) to (i) remove transient time in the signal, (ii) remove baseline wander, and (iii) remove powerline noise. To remove transient, Module 400 may remove the x seconds of the signal (e.g., 10 seconds), e.g., to remove the signal period of a high likelihood of motion that can be present and associated with electrode settling and contact. To remove baseline wander, Module 400 may employ a forward-reverse high-pass filter (e.g., a 2nd order 0.67 Hz forward-reverse high-pass filter) or other like phase linear filters. To remove power line noise, Module 400 may employ a band-stop filter (e.g., 2^{nd}-order band-stop filter with half-power frequencies of 59 and 61 Hz coupled with a Butterworth IIR filter).

Periodic Windowing. Module 400 may segment (804) the signal into windows of sub-signals. A rectangular window may be first employed, having a duration of x (e.g., 70 seconds). To reduce spectral leakage, the 70-second sub-signal is further reduced using a landmark detection operation (e.g., Pan Tompkins detector). The landmark detector may delineate the cardiac signal to identify the ventricular repolarization termination (VRT) and atrial depolarization onset (ADO) fiducial points. A search window of 50 seconds may be set to find the first VRT, and last ADO in the windowed signal (e.g., 70-second sub-signal) and the maximum of VRT, and the minimum of ADO in all three channels are assigned to rectangular window edges. This operation may be particularly beneficial for coherence analysis as the sub-signal should have the same number of data points in all the channels. Use of a search window (e.g., a 50-second search window) for spectral analysis can lead to a resolution of 0.02 Hz:

Cumulative Spectral Power Calculation. Module 400 may employ the Welch's operation (also referred to as a periodogram), and like operations, for the spectral analysis to calculate power spectral density, including, for example, the cumulative power of X, Y, and Z channels. The Welch's operation is described in Equation 1.

$${\hat{S}}_{x}^{W} ( {w}_{k} ) {=}^{Δ} \frac{1}{K} {\sum }_{m = 0}^{K - 1} {P}_{{x}_{m} , M} ( {w}_{k} ) \qquad ( Equation 1 )$$

In Equation 1, Pₓ_{ₘ}_{,M}(Wₖ) is the periodogram of an m^{th} block of a zero-padded frame from a signal x and is given by Equation 2. The m^{th} window frame is denoted as

$${x}_{m} ( n ) {=}^{△} w ( n ) x ( n + mR ) ,$$

n=0, 1, . . . , M−1, m=0, 1, . . . , K−1, where R is a defined window hop size, and K is the number of available frames. When w(n) is a rectangular window, the periodograms are formed from non-overlapping successive blocks of data. For other window types, the analysis frames typically overlap.

$${P}_{{x}_{m} , M} ( {w}_{k} ) = \frac{1}{M} {\vert {FFT}_{N , k ( {x}_{m} )} \vert}^{2} {=}^{△} \frac{1}{M} {\vert {\sum }_{n = 0}^{N - 1} {x}_{m} ( n ) {e}^{- j 2 π nk / N} \vert}^{2} \qquad ( Equation 2 )$$

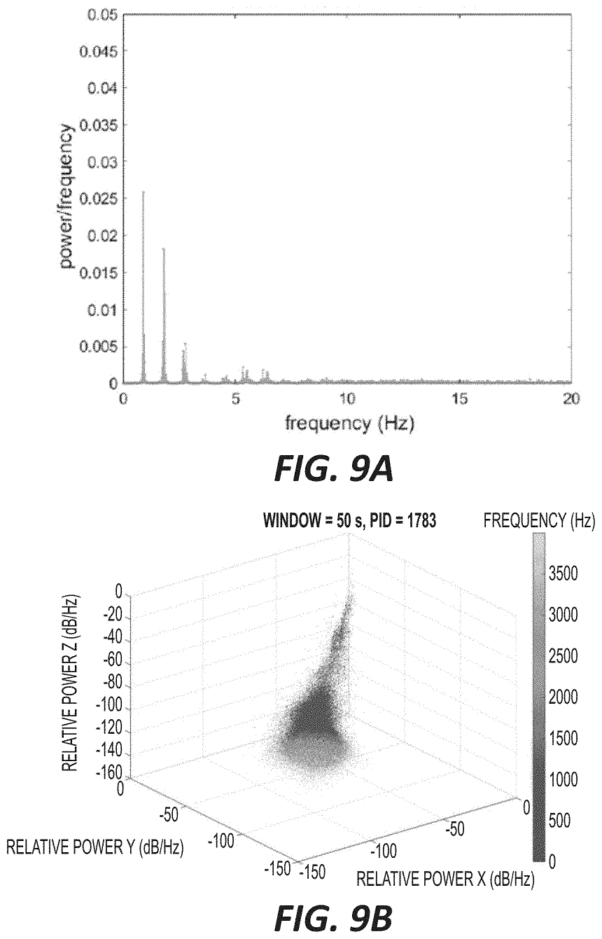

Module 400 may implement a periodogram to calculate the cumulative power of a given biophysical signal using an FFT operator with a 10% cosine fraction Tukey window. FIG. 9A shows an example FFT periodogram used to calculate the power spectral density of a biophysical signal in accordance with an illustrative embodiment. The periodogram in FIG. 9A was preprocessed for baseline removal, transient time removal, and powerline filtering, periodic windowing, and spectral windowing (using a Tukey window with a 0.1 cosine fraction).

Welch's operation is a Fourier-based algorithm that estimates the power spectrum by dividing the time signal into successive blocks and forming the periodogram for each block. The averaging of that result is used to obtain a statistical representation of the power spectrum. In the calculation of the power spectrum, it is preferred to maximize the block size (number of data points in the segment of signal) to maximize spectral resolution; however, at the same time, using more blocks provides more data for averaging, and hence, greater spectral stability. Accordingly, the Welch's operation can reduce the leakage and reduce the frequency resolution, which could be unfavorable. Tuning the number of blocks may be beneficial when the Welch's operation is applied to the data. Tuning can be done depending on the required frequency resolution within the frequency range of interest (e.g. it could vary for different diseases) and the characteristics of the acquired signal (length and the quality of the signal).

Generally, spectral leakage results from non-linearities that are introduced prior to and/or during a transformation which can result in the dissipation of the power to the neighboring frequencies that may not be present in the original data. Spectral leakage may be observed as new frequency components, aside from the frequency component in the original data, introduced into the result during a transformation operation such as sampling frequency, windowing, and filtering the signal [12]. For example, applying Fourier transform to an infinite non-stationary time series whose duration is limited by the acquisition time (rectangular windowing) can cause non-linear operations on data [12]. In stationary periodic signals, leakage can occur during Fourier transform (FT for continuous and DFT for discrete time-signal) by windowing the signal with a non-integer number of periods in the signal. The same issue arises for quasi-periodic and non-periodic stationary signals.

Periodic windowing operations (as used in the Welch's operation) may be used to reduce the spectral leakage in periodic signals and refers to the trimming of a signal to a segment such that the duration of the windowed signal is a multiple of the dominant period in the signal. While periodic windowing can effectively reduce spectral leakage, it can mainly be applied to periodic signals with repeated patterns. Welch's operation also increases the spectral resolution (δf), which is the smallest measurable increment in the frequency domain, to reduce spectral leakage. Spectral resolution often has an inverse relationship to the duration of the signal (ts), i.e., δf(Hz)=1/tₛ(s). Examples of windowing operations that may be used include Hann, Hamming, Rectangular (no window), Blackman, Gaussian, and Tukey.

Total Spectral Power Calculation. Module 400 may determine the total spectral power in the X, Y, and Z channels (shown as the “total_power” feature in Table 1) by determining the power spectral density for each of the X, Y, and Z channels and summing them together in three-dimensional space below the Nyquist cut-off frequency, e.g., as shown in Equation 3.

$${P}_{total , Nyquist} = δ {f}_{k} {\sum }_{f = 0}^{f = {f}_{s} / 2} {P}_{x} ( f ) + {P}_{y} ( f ) + {P}_{z} ( f ) \qquad ( Equation 3 )$$

In Equation 3, (δfₖ) has a fixed resolution.

Relative Spectral Power Calculation. Module 400 may determine the relative spectral power in the X, Y, and Z channels as a ratio of the power of the channel over the total power of the channels in three-dimensional space below the Nyquist cut-off frequency, as shown in

$${S}_{x , y , z , rel} ( f ) = \frac{{P}_{x , y , z} ( f )}{{\sum }_{f = 0}^{f = {f}_{s} / 2} {P}_{x} ( f ) + {P}_{y} ( f ) + {P}_{z} ( f )} \qquad ( Equation 4 )$$

While the power is calculated for the range of frequency between 0 to Nyquist frequency (fₛ/2), it has been observed through analysis that most of the biopotential power is concentrated in frequencies below 50 Hz. From the observation, the frequency content of the ventricular repolarization wave contributes to the lowest frequency of biopotential signals, ranging from 0 to 10 Hz, with some overlay with atrial depolarization/repolarization waves, characterized by 5-30 Hz frequencies. This is consistent with publications that report that the ventricular depolarization wave contains most of the biopotential energy, which normally demonstrates itself within 8-50 Hz frequencies [14].

FIG. 9B shows the power density distribution in three-dimensional space for a random subject. As observed in the example, the power distribution in higher frequencies (f>50 Hz) is normal (i.e., funnel-shaped in log scale), which is mainly due to noise behavior. This distribution could cause high uncertainty in the spectral and coherence features, and thus, signals are filtered with a cut-off frequency of 50 Hz to attenuate the high-frequency content of the signal. By narrowing the frequency band below 50 Hz, the power spectral and coherence analysis's main focus is on the low-moderate frequency component of the biopotential signals. Having the filtered signal, relative power in channel X, for example, can be calculated by Equation 5.

$${P}_{x , rel} ( f ) = \frac{{P}_{x} ( f )}{{\sum }_{f = 0}^{f = 50 H z} {P}_{x} ( f ) + {P}_{y} ( f ) + {P}_{z} ( f )} \qquad ( Equation 5 )$$

From experimentation, it is observed that 2% power is lost when signals are filtered below a 50 Hz cut-off frequency. Indeed, the analysis using Equation 5 preserves the frequency components of a biophysical signal such as biopotential cardiac signals for spectral analysis.

### Example #2—Power Spectral Features or Parameters (Coherence)

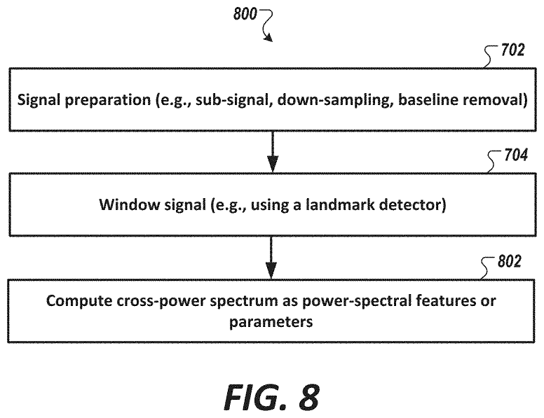

FIG. 5 illustrates, as the second of three example feature or parameter categories, an example coherence analysis feature computation module 500 configured to determine cross-power spectral attributes between biophysical signals. FIG. 8, discussed below, shows an example method 800 of operation of Module 500.

Table 2 shows an example set of 10 types of extractable cross-power spectral features and their corresponding description to provide up to 37 features (see Table 3) for an example set of biophysical signals. In Table 2, 5 feature types (“sum_coherence,” “std_coherence,” “skew_coherence,” “kurt_coherence” and “entropy_coherence”) have been observed to have significant utility in the assessment of the presence or non-presence of at least one cardiac disease or condition—specifically, the determination of presence or non-presence of elevated LVEDP. In Table 2, at least one feature type (“sum_coherence”) has also been observed to have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 8A, and Tables 9A and 9B, respectively.

TABLE 2

| Feature name | Description |
|---|---|
| “sum_coherence” or “cohSum”*, ** | Cumulative coherence (magnitude |
| | squared coherence) between two |
| | biophysical signals |
| “total_sum_coherence”¹ | Total cumulative coherence for a |
| | given set of biophysical signals |
| “mean_coherence” or “cohMean” | Coherence mean between two |
| | biophysical signals |
| “median_coherence” or | Median of coherence distribution |
| “cohMedian” | between two biophysical signals |
| “std_coherence” or “cohStd”* | Standard deviation of coherence |
| | distribution between two |
| | biophysical signals |
| “var_coherence” or “cohVar” | Variance of coherence distribution |
| | between two biophysical signals |
| “skew_coherence” or “cohSkew”* | Skewness of coherence distribution |
| | between two biophysical signals |
| “kurt_coherence” or “cohKurt”* | Kurtosis of coherence distribution |
| | between two biophysical signals |
| “entropy_coherence” or | Entropy of coherence distribution |
| “cohEntropy”* | between two biophysical signals |
| “SSELognormal_coherence” | Summed Square of residuals |
| | (error) between coherence of |
| | biophysical signals due to model |
| | fitting |

¹= single parameter

Table 3 shows a summarized set of 37 cross-power spectral-based features (“Parameters”) between two biophysical signals (e.g., between cardiac channels X and Y, channels X and Z, channels Y and Z, and between PPG signal, channels #1 and #2). The features in Tables 2 and 3 can be generated for each signal or waveform unless indicated to be a single parameter “**”.

TABLE 3

| Parameters | Signals |
|---|---|
| “sum_coherence” or “cohSum” | Orth_1_2 (cardiac signal, X |
| | and Y channels) |
| “total_sum_coherence** | Orth_1_3 (cardiac signal, X |
| | and Z channels) |
| “mean_coherence” or “cohMean” | Orth_2_2 (cardiac, signal, Y |
| | and Z channels) |
| “median_coherence” or “cohMedian” | PPG signal channels #1 and #2 |
| “std_coherence” or “cohStd”* | |
| “var_coherence” or “cohVar” | |
| “skew_coherence” or “cohSkew” | |
| “kurt_coherence” or “cohKurt” | |
| “entropy_coherence” or “cohEntropy”* | |
| “SSELognormal_coherence” | |

** refers to a single parameter

FIG. 8 shows a method 800 to generate power spectral-based features or parameters, e.g., as performed by the coherence analysis feature computation module 600 of FIG. 6, in accordance with an illustrative embodiment, which can be used wholly, or partially, to generate power spectral-based features or parameters and its outputs to be used in machine-learned classifier to determine a metric associated with a physiological system of a subject under study. To determine the features of Table 2, Module 600 is configured, in some embodiments, to (i) pre-process (702) the acquired biophysical signal, (ii) window (704) the signals to improve spectral leakage losses, and (iii) determine (802) coherence of the windowed signals as the cross-power spectral features or parameters.

Cumulative Coherence. While power spectral analysis focuses on the power distribution of a signal in the frequency domain, coherence is a measure of cross-spectral characteristics across two signals. To determine the sum coherence (also referred to as cumulative coherence) between two biophysical signals, Module 500 may calculate the magnitude squared coherence (MSC) of two time-signals x and y (C_{xy}) as shown in Equation 6A.

$${C}_{xy} ( f ) = \frac{{\vert {P}_{x y} ( f ) \vert}^{2}}{{P}_{x} ( f ) {P}_{y} ( f )} , {P}_{x} ( f ) \neq 0 and {P}_{y} ( f ) \neq 0 \qquad ( Equation 6 A )$$

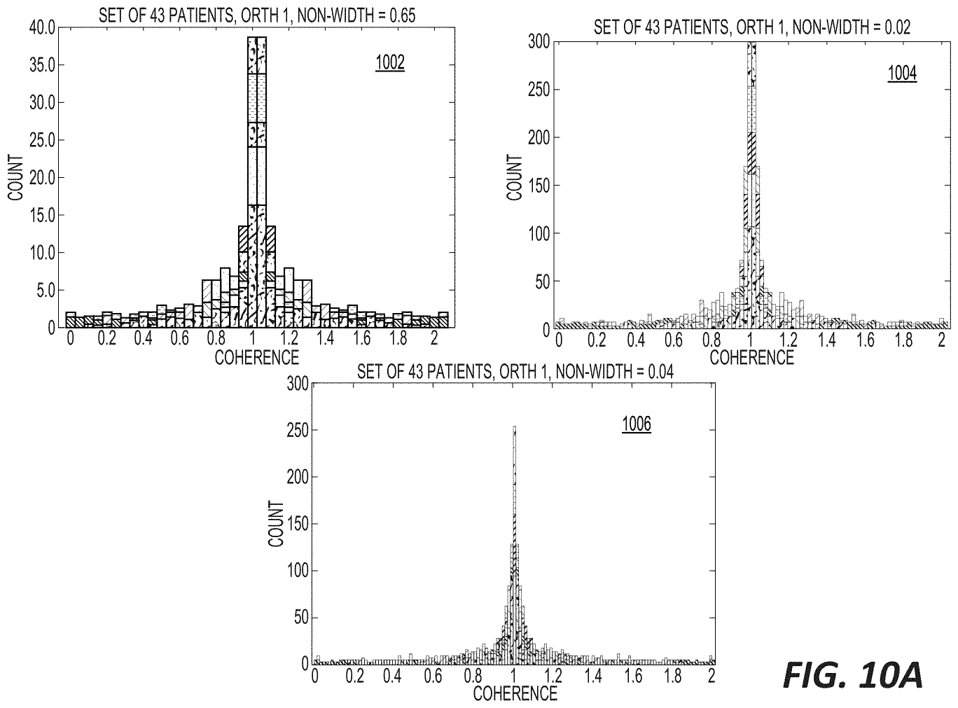

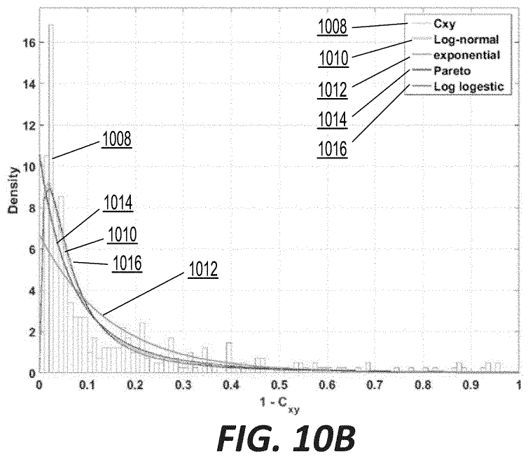

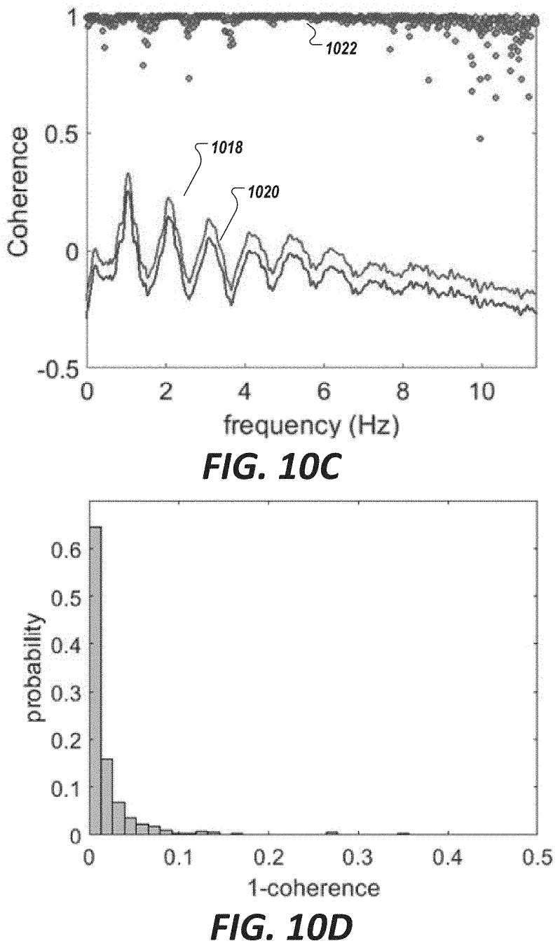

In Equation 6A, P is the signal power representation in the frequency domain (also referred to as power spectral density). FIG. 10A shows C_{xy} for 43-set patients with a different number of bins: 20 bins are shown in 1002, 50 bins are shown in 1004, and 100 bins are shown in 1006. After visually assessing the results in FIG. 10A, the 100 bins was selected as it appears to capture the distribution with enough level of details required for distribution analysis.

Equation 6B shows the magnitude squared coherence (MSC) for two photoplethysmographic signals.

$$C ( f ) = \frac{{\vert {P}_{R ed - IR} ( f ) \vert}^{2}}{{P}_{R e d} ( f ) {P}_{IR} ( f )} , {P}_{R e d} ( f ) \neq 0 and {P}_{IR} ( f ) \neq 0 \qquad ( Equation 6 B )$$

FIG. 10C shows an example of cumulative coherence (1022) determined from two FFTs photoplethysmographic signals (1018, 1020) as plotted against frequency.

Module 500 may then calculate the sum coherence (e.g., cumulative coherence) for a given channel pair x, y, z corresponding to channels XZ, XY, and YZ as shown in Equation 7.

$${C}_{c u m , i} = {\sum }_{{f}_{k} = 0}^{{{f}_{k} = 50 Hz}^{}} {c}_{i} ( f ) i = x , y , z \qquad ( Equation 7 )$$

The Welch's operation may be applied to the periodically windowed biopotential signals. To increase the statistical stability of the coherence spectra, x successive blocks (e.g., 4 successive blocks) with Hamming window (no overlap) may be used.

Total Sum Coherence. Module 500 may calculate the total cumulative coherence as shown in Equation 8.

$${C}_{cum} = {\sum }_{{f}_{k} = 0}^{{{f}_{k} = 50 Hz}^{}} {c}_{x} ( f ) + {c}_{y} ( f ) + {c}_{z} ( f ) \qquad ( Equation 8 )$$

Statistics of Coherence. To generate the coherence distribution feature (mean, median, kurtosis, standard deviation, variance, entropy, lognormal fit, of distribution of coherence), Module 600 may first calculate a coherence density distribution (CDD) per Equation 9.

$$CDD ( {C}_{i} ) = \frac{{F}_{f} ( {c}_{i} )}{{(}^{1} {/}_{{N}_{b i n}} ) {\sum }_{w = 0}^{w = {N}_{b i n}} {F}_{w} ( {c}_{i} )} \qquad ( Equation 9 )$$

In Equation 9, F_{ω} is the frequency of occurrences, and N_{bin} is the number of bins. Module 600 may then characterize the CDD distribution by statistical assessments such as mean μ, median, standard deviation a, skewness, kurtosis, entropy, and lognormal fit.

Kurtosis is a statistical measure that defines how heavily the tails of a given distribution differ from the tails of a normal distribution. Kurtosis of a distribution may be defined as k=E(x−μ)⁴/σ⁴, where μ is the mean of a distribution x, σ is the standard deviation of x, and E( ) is the expected value of x−μ.

Skewness is a measure of the asymmetry of the data around the sample mean. If skewness is negative, the data spread out more to the left of the mean than to the right. If skewness is positive, the data spread out more to the right. The skewness of the normal distribution (or any perfectly symmetric distribution) is zero. The skewness of a distribution may be defined as s=E(x−μ)³/σ³, where μ is the mean of a distribution x, σ is the standard deviation of x, and E( ) represents the expected value of the x−μ.

Entropy may be calculated per Equation 10.

$${E}_{i} = {\sum }_{w = 0}^{w = {N}_{b i n}} {P}_{w} ( {c}_{i} ) log {P}_{w} ( {c}_{i} ) i = x , y , z \qquad ( Equation 10 )$$

In Equation 10, the probability of cᵢ is defined per Equation 11.

$${P}_{w} ( {c}_{i} ) = \frac{{F}_{w} ( {c}_{i} )}{{\sum }_{w = 0}^{w = {N}_{b i n}} {F}_{w} ( {c}_{i} )} \qquad ( Equation 11 )$$

Lognormal distribution. Module 600 may employ a lognormal distribution to fit the CDD as the “SSELognormal_coherence” feature. FIG. 10B shows a coherence density distribution (CDD) 1008 calculated with a number of distribution functions fitted to the data, including a lognormal fit 1010, an exponential fit 1012, a pareto fit 1014, and a log-logistic fit 1016. In FIG. 10B, lognormal appears to be the best distribution representing the data. As shown in FIG. 10B, other distribution fits may be used, including, for example, but not limited to, exponential fit, pareto fit, and log-logistic fit. FIG. 10D shows the coherence density distribution for a set of photoplethysmographic signals to which a similar fit may be determined.

The lognormal distribution is a continuous probability distribution of a random variable in which the distribution is normally (Gaussian) distributed. Additional descriptions of the lognormal distribution may be found in [15], [16]. Module 600 may determine the residue (unexplained) as the difference between a model (explained), and the actual data (observation) and the sum of squared residuals (SSR) are used as a feature describing the measure of goodness of fit.

### Example #3—Power Spectral Features or Parameters (Frequency Modes)

FIG. 6 illustrates, as the third of three example feature or parameter categories, an example coherence analysis feature computation module 600 configured to determine cross-power spectral attributes between biophysical signals. FIG. 7B shows an example method 700b of Module 600.

Table 4 shows an example set of 10 types of extractable power spectral features and their corresponding description, which can provide up to 18 features for an example set of biophysical signals (Table 5). In Table 4, 7 feature types (“DRM1”, “LDM1”, “LDM2”, “MTFreq”, “pM1”, “pRatioM1”, and “pRatioM2”) have been observed to have significant utility in the assessment of the presence or non-presence of at least one cardiac disease or condition—specifically, the determination of presence or non-presence of elevated LVEDP. In Table 4, 4 feature types (“DRM1,” “LDM2,” “pM1,” and “pRatioM1”) have also been observed to have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 8B and Table 9, respectively.

TABLE 4

| Feature Name | Description |
|---|---|
| DRM1*,** | Decay Rate Mode “1” of the power spectral of a |
| | biophysical signal (Slope of the Mode 1 fit line) |
| LDM1* | Linear Decay Mode “1” of the power spectral of a |
| | biophysical signal (R-Square of the fit) |
| DRM2 | Decay Rate Mode “2” of the power spectral of a |
| | biophysical signal (Slope of the Mode 2 fit line) |
| LDM2*,** | Linear Decay Mode “2” of the power spectral of a |
| | biophysical signal (R-Square of the fit) |
| MTFreq* | Mode Transition Frequency (Intersection |
| | frequency of a mode “1” peak and a mode “2” |
| | peak) |
| pM1*,** | Power Mode “1” of the power spectral of a |
| | biophysical signal (Sum signal power for f < |
| | MTFreq) |
| pM2 | Power Mode “2” of the power spectral of a |
| | biophysical signal (Sum signal power for MTF < |
| | f < 30 Hz) |
| pRatioM1M2 | Power Ratio Mode “1” to Mode “2” in the power |
| | spectral of a biophysical signal (pM1_Red/ |
| | pM2_Red) |
| pRatioM1*,** | Power Ratio Mode “1” of the spectral power of |
| | the 1^{st} signal to the spectral power of Mode |
| | “1” of the 2^{nd} signal (pM1_Red/ |
| | pM1_IR) |
| pRatioM2* | Power Ratio Mode “2” of the spectral power of |
| | the 1^{st} signal to the spectral power of the |
| | 1^{st} signal of Mode “1” of the 2^{nd} signal |
| | (pM2_Red/pM2_IR) |
| pRatioMax_Red_IR | Power Ratio Maximum between spectral power of |
| | biophysical signals (P_{fundamental Frequency Red}/ |
| | P_{fundamental Frequency IR}) |

Table 5 shows a summarized set of 18 power spectral-based features (“Parameters”) for a given photoplethysmographic signal. In the example, 7 parameters can be generated for each of the two acquired PPG waveforms to provide 14 features, and 4 features can be generated from the combination of both PPG waveforms. Similar features or parameters may be generated and computed by Module 600 for cardiac signals and other biophysical signals.

TABLE 5

| Parameters | Signals |
|---|---|
| DRM1 | PPG signal channel #1 (red) |
| LDM1 | PPG signal channel #2 (IR) |
| DRM2 | |
| LDM2 | |
| MTFreq* | |
| pM1 | |
| pM2 | |
| pRatioM1M2 | |
| pRatioM1_Red_IR* | |
| pRatioM2_Red_IR* | |
| pRatioMax_Red_IR* | |

*= 1 parameter only

FIG. 7B shows a method 700b to generate power spectral-based features or parameters for a photoplethysmographic signal, e.g., as performed by the power spectral analysis feature computation module 600 of FIG. 6, in accordance with an illustrative embodiment, which can be used wholly, or partially, to generate power spectral-based features or parameters and its outputs to be used in machine-learned classifier to determine a metric associated with a physiological system of a subject under study. To determine the features of Table 5, Module 600 is configured, in some embodiments, to (i) pre-process (702) the acquired biophysical signal, (ii) window (704) the signals (to improve spectral leakage losses), (iii) determine (708) the power spectrum of the windowed signals, (iv) determine (710) a transition frequency as an intersection of two fitted trendline to a low and a high-frequency portion of the power spectrum, and compute (712) the power-spectral features or parameters using portions of the power spectrum as defined by the transition frequency.

Power Spectrum Calculation. Module 600 may employ the Welch operation above to calculate the power spectrum (operation 708), as well as other later operations. The power of a discrete-time signal, y(n), in the time and frequency domain can be calculated by Equations 12 and 13.

$${P}_{y} = \frac{1}{T} 〈 y ( n ) , y ( n ) 〉 = \frac{1}{N} {{\sum }^{N}}_{0} {\vert y ( n ) \vert}^{2} \qquad ( Equation 12 )$$

$${P}_{Y} = \frac{1}{N} {\sum }_{0}^{N} {\vert Y ( f ) \vert}^{2} \qquad ( Equation 13 )$$

In Equation 13, Y(f) is the continuous Fourier transform of signal y(t).

Transition Frequency Calculation. Having the sub-signal periodically sampled, the spectral analysis may also be performed using Welch operation. The Welch operation is a Fourier-based algorithm that estimates the power spectrum by dividing the time signal into successive blocks, forming the periodogram for each block, and averaging the results to obtain a statistical representation of the power spectrum. The Welch operation may set for a number of discrete Fourier transform (DFT) points to estimate a Welch low-resolution power spectrum density (WLR-PSD) and a Welch high-resolution power spectrum density (WHR-PSD). Table 6 lists the parameters used to calculate Welch's power spectral density estimate.

TABLE 6

| Parameter | Value |
|---|---|
| Segment duration | 180 seconds |
| Number of segments | 10 |
| Segment overlap | 0.5 (between 0 and 1) |
| N_{DFT-high resolution} | N_{segment} = (Segment duration) × (Fₛ) |
| N_{DFT-low resolution} | (½) N_{segment} |

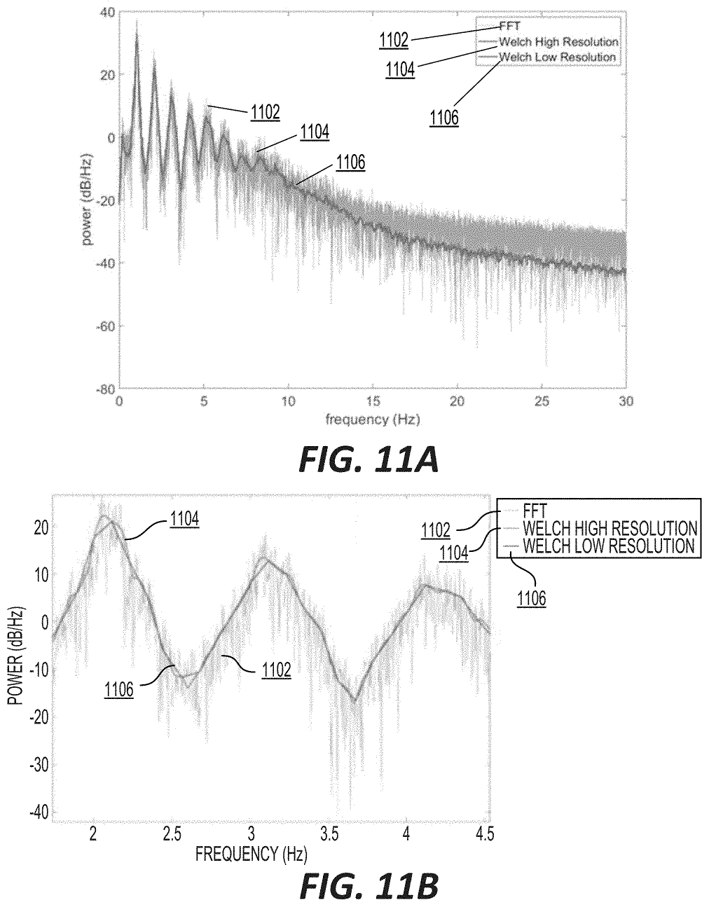

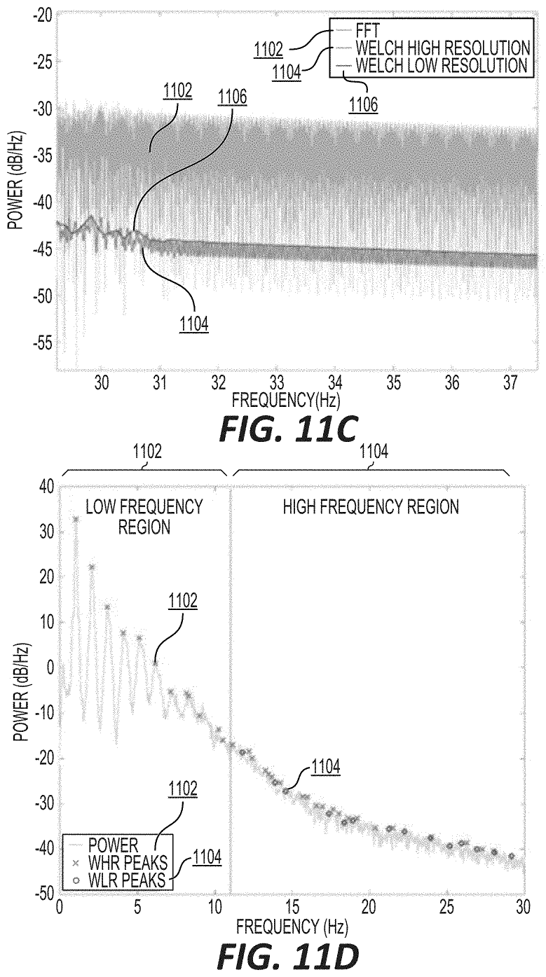

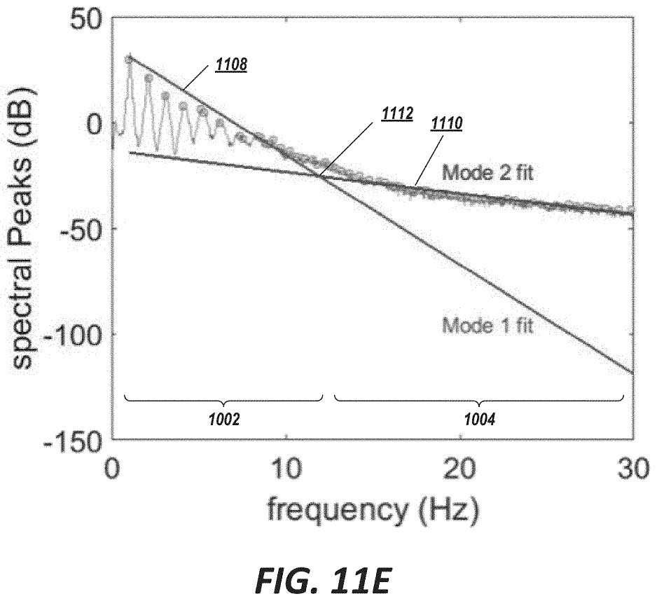

FIGS. 11A-11E show various aspects of power spectral analysis of the method of FIG. 7B in accordance with an illustrative embodiment. Specifically, FIGS. 11A-11C shows an example power spectrum density for a sample subject using FFT (1002) along with computed WHR-PSD (1104) and WLR-PSD (1106). In FIG. 11A, it can be observed that the Welch spectrums are smooth with i) low leakage and ii) distinct spectral peaks as compared to the FFT spectrum. In FIG. 11A, the Welch operation estimated lower power at higher frequencies (e.g., f>10 Hz). The high-frequency power is mainly associated with random high-frequency noises in the signal, which has been mitigated through the averaging of the power in the Welch operation.

FIGS. 11B and 11C, respectively, show a low-resolution and high-resolution Welch spectrum of FIG. 11A at low and high-frequency ranges. In FIGS. 11B and 11C, while WHR-PSD follows the FFT spectrum with higher resolution at lower frequencies, it can be observed that WHR-PSD tries to capture the noise content at higher frequencies. In contrast, the WLR_PSD appears to have less frequency resolution at lower frequencies but can smooth the noise spectrum at high frequencies.

Using WHR-PSD (1106) and the WLR-PSD (1104), Module 600 can perform robust spectral peak analysis at low frequencies and across a wide range of frequencies. Spectral peaks may be detected while the subharmonics peaks are removed. Because the WHR (1106) is developed to identify the high-frequency content of the signal, a peak finder parameter may be set to ensure only peaks are selected.

FIG. 11D shows the PPG power spectral analysis feature computation module 600 in being able to separate the PSD into two regions: “Mode 1” (1102) comprising the low-frequency region and “Mode 2” (1104) comprising the high-frequency region with the first 20 low-frequency peaks filtered.

FIG. 11E shows the operation of PPG power spectral analysis feature computation module 600 to quantify i) the rate of decay 1108 in power with respect to frequency and ii) a linear regression line 1110 that is fitted to the spectral fit in each mode in accordance with an illustrative embodiment. The transition point 1112 between Mode “1” 1102 and Mode “2” 1104 may be calculated by sequentially adding (from low to high frequency) spectral peaks of WHR to its first ten peaks and tracking the fitting quality. The regression line with the highest R-squared may be considered as the Mode “1” fit. In FIG. 11E, Mode “2” fit was generated similarly by adding the spectral peaks of WLR (1102) sequentially (from high to low frequency) to its last 20 peaks and pick the fit with the highest R-squared.

In FIG. 11E, the rate of decay 1108 may be extracted, as the slope, from each of two or more photo photoplethysmographic signals and for each of the two modes (“DRM1_Red”, “DRM2_Red”, “DRM1_IR”, and “DRM2_IR”). The R-square of the fit for the slope may also be extracted for each of the two or more photo photoplethysmographic signals and for each of the two modes (“LDM1_Red”, “LDM2_Red”, “LDM1_IR”, “LDM2_IR”). The frequency value of the transition point 1106 may also be extracted (“MTFreq”).

In addition, once the transition point 1112 is defined, Module 600 can also compute the sum signal power for i) frequencies below the transition point 1106 for each of the two or more photo photoplethysmographic signals (“pM1”) and ii) frequencies above the transition point 1112 for each of the two or more photo photoplethysmographic signals (“pM2” features). Module 600 may also compute i) the ratios between the modes (“pRatioM1M2” features) and ii) the ratios between the signals (“pRatioM1” and “pRatioM2” features). In addition, Module 600 may compute the ratio value between the fundamental frequency defined in each of the two signals.

### Experimental Results and Examples

Several development studies have been conducted to develop feature sets and, in turn, algorithms that can be used to estimate the presence or non-presence, severity, or localization of diseases, medical conditions, or an indication of either. In one study, algorithms were developed for the non-invasive assessment of abnormal or elevated LVEDP. As noted above, abnormal or elevated LVEDP is an indicator of heart failure in its various forms. In another development study, algorithms and features were developed for the non-invasive assessment of coronary artery disease.

As part of these two development studies, clinical data were collected from adult human patients using a biophysical signal capture system and according to protocols described in relation to FIG. 2. The subjects underwent cardiac catheterization (the current “gold standard” tests for CAD and abnormal LVEDP evaluation) following the signal acquisition, and the catheterization results were evaluated for CAD labels and elevated LVEDP values. The collected data were stratified into separate cohorts: one for feature/algorithm development and the other for their validation.

Within the feature development phases, features were developed, including the power spectral-based features or parameters, to extract characteristics in an analytical framework from biopotential signals (as an example of the cardiac signals discussed herein) and photo-absorption signals (as examples of the hemodynamic or photoplethysmographic discussed herein) that are intended to represent properties of the cardiovascular system. Corresponding classifiers were also developed using classifier models, linear models (e.g., Elastic Net), decision tree models (XGB Classifier, random forest models, etc.), support vector machine models, and neural network models to non-invasively estimate the presence of an elevated or abnormal LVEDP. Univariate feature selection assessments and cross-validation operations were performed to identify features for use in machine learning models (e.g., classifiers) for the specific disease indication of interest. Further description of the machine learning training and assessment are described in U.S. Provisional Patent Application No. 63/235,960, filed Aug. 23, 2021, entitled “Method and System to Non-Invasively Assess Elevated Left Ventricular End-Diastolic Pressure” having attorney docket no. 10321-048pv1, which is hereby incorporated by reference herein in its entirety.

The univariate feature selection assessments evaluated many scenarios, each defined by a negative and a positive dataset pair using a t-test, mutual information, and AUC-ROC evaluation. The t-test is a statistical test that can determine if there is a difference between two sample means from two populations with unknown variances. Here, the t-tests were conducted against a null hypothesis that there is no difference between the means of the feature in these groups, e.g., normal LVEDP vs. elevated (for LVEDP algorithm development); CAD− vs. CAD+ (for CAD algorithm development). A small p-value (e.g., <0.05) indicates strong evidence against the null hypothesis.

Mutual information (MI) operations were conducted to assess the dependence of elevated or abnormal LVEDP or significant coronary artery disease on certain features. An MI score greater than one indicates a higher dependency between the variables being evaluated. MI scores less than one indicates a lower dependency of such variables, and an MI score of zero indicates no such dependency.

A receiver operating characteristic curve, or ROC curve, illustrates the diagnostic ability of a binary classifier system as its discrimination threshold is varied. The ROC curve may be created by plotting the true positive rate (TPR) against the false positive rate (FPR) at various threshold settings. AUC-ROC quantifies the area under a receiver operating characteristic (ROC) curve—the larger this area, the more diagnostically useful the model is. The ROC, and AUC-ROC, value is considered statistically significant when the bottom end of the 95% confidence interval is greater than 0.50.

Table 7 shows an example list of the negative and a positive dataset pair used in the univariate feature selection assessments. Specifically, Table 7 shows positive datasets being defined as having an LVEDP measurement greater than 20 mmHg or 25 mmHg, and negative datasets were defined as having an LVEDP measurement less than 12 mmHg or belonging to a subject group determined to have normal LVEDP readings.

TABLE 7

| Negative Dataset | Positive Dataset |
|---|---|
| ≤12 (mmHg) | ≥20 (mmHg) |
| ≤12 (mmHg) | ≥25 (mmHg) |
| Normal LVEDP | ≥20 (mmHg) |
| Normal LVEDP | ≥25 (mmHg) |

Tables 8A and 8B each shows a list of power spectral-based features having been determined to have utility in estimating the presence and non-presence of elevated LVEDP in an algorithm executing in a clinical evaluation system. The features of Tables 8A and 8B and corresponding classifiers have been validated to have clinical performance comparable to the gold standard invasive method to measure elevated LVEDP.

TABLE 8A

| Feature_name | t-test | AUC | MI |
|---|---|---|---|
| coherence_entropy_XZ | 0.0448 | n/s | 1.2166 |
| ssr_XZ | 0.0101 | 0.5151 | n/s |
| ssr_signal | 0.0076 | 0.5219 | n/s |
| coherence_std_XZ | 0.0053 | 0.5120 | n/s |
| coherence_kurt_YZ | 0.0069 | 0.5122 | n/s |
| coherence_skew_YZ | 0.0149 | 0.5206 | n/s |
| coherence_skew_XZ | 0.0081 | 0.5190 | n/s |
| coherence_kurt_XZ | 0.0049 | 0.5252 | n/s |
| cohKurt_PPG | 0.0349 | n/s | 1.3560 |
| cohEntropy_PPG | 0.0172 | n/s | n/s |
| cohSkew_PPG | 0.0450 | n/s | n/s |
| cohSum_PPG | 0.0050 | n/s | n/s |

FA scenario = LVEDP <= 12 (N = 246) vs >=20 (N = 209)

TABLE 8B

| Feature_name | t-test | AUC | MI |
|---|---|---|---|
| pM1_Red | 0.0269 | 0.5173 | n/s |
| pM1_IR | n/s | 0.5112 | n/s |
| LDM1_Red* | n/s | 0.7268 | n/s |
| DRM1_Red* | 0.0267 | 0.5453 | 1.9094 |
| LDM2_Red | 0.0268 | 0.5317 | n/s |
| LDM2_IR | 0.0018 | 0.5350 | n/s |
| pRatioM1M2_IR | 0.0113 | 0.5252 | n/s |
| pRatioM1M2_Red | 0.0266 | 0.5184 | n/s |
| TMFreq* | 0.0290 | n/s | 1.1924 |

FA scenario = LVEDP <= 12 (N = 246) vs >=20 (N = 209)
*= LVEDP <= 20 (N = 95) vs CADHealth G1 (N = 122)

Tables 9A and 9B each shows a list of power spectral-based features having been determined to have utility in estimating the presence and non-presence of significant CAD in an algorithm executing in a clinical evaluation system. The features of Tables 9A and 9B and corresponding classifiers have been validated to have clinical performance comparable to the gold standard invasive method to measure CAD.

TABLE 9A

| | t-test p-value | AUC | MI |
|---|---|---|---|
| cohSum_PPG | 0.0349 | n/s | 1.3076 |
| pM1_IR | 0.0088 | 0.5059 | n/s |
| pRatioM1_Red_IR | 0.0135 | n/s | 1.0850 |
| LDM2_Red | 0.0407 | n/s | n/s |
| pM1_IR | 0.0154 | n/s | n/s |
| DRM1_Red | n/s | n/s | 1.0054 |

FA scenario = significant CAD (e.g., defined as >70% blockage and/or FFR < 0.8) (N = 464; 232 CAD positives and 232 CAD negatives (½ single and ½ multi-vessel disease) (½ are males and ½ are females)

TABLE 9B

| | t-test p-value | AUC | MI |
|---|---|---|---|
| cohSum_YZ | 0.0446 | 0.5035 | n/s |
| ssr_XZ | 0.0487 | n/s | n/s |
| power_std_Y | n/s | n/s | 1.0800 |
| cohKurt_YZ | n/s | n/s | 1.0061 |
| ssr_signal | n/s | n/s | 1.0107 |

FA scenario = significant CAD (e.g., defined as >70% blockage and/or FFR < 0.8) (N = 464; 232 CAD positives and 232 CAD negatives (½ single and A multi-vessel disease) (½ are males and ½ are females)

The determination that certain power spectral-based features have clinical utility in estimating the presence and non-presence of elevated LVEDP or the presence and non-presence of significant CAD provides a basis for the use of these power spectral-based features or parameters, as well as other features described herein, in estimating for the presence or non-presence and/or severity and/or localization of other diseases, medical condition, or an indication of either particularly, though not limited to, heart disease or conditions described herein.

The experimental results further indicate that intermediary data or parameters of power spectral-based features also have clinical utility in diagnostics as well as treatment, controls, monitoring, and tracking applications.

Example Clinical Evaluation System

FIG. 12A shows an example clinical evaluation system 1200 (also referred to as a clinical and diagnostic system) that implements the modules of FIG. 1 to non-invasively compute power spectral-based features or parameters, along with other features or parameters, to generate, via a classifier (e.g., machine-learned classifier), one or more metrics associated with the physiological state of a patient or subject according to an embodiment. Indeed, the feature modules (e.g., of FIGS. 1, 4-8) can be generally viewed as a part of a system (e.g., System 1200 the clinical evaluation system 1200) in which any number and/or types of features may be utilized for a disease state, medical condition, an indication of either, or combination thereof that is of interest, e.g., with different embodiments having different configurations of feature modules. This is additionally illustrated in FIG. 12A, where the clinical evaluation system 1200 is of a modular design in which disease-specific add-on modules 1202 (e.g., to assess for elevated LVEDP or mPAP, CAD, PH/PAH, abnormal LVEF, HFpEF, and others described herein) are capable of being integrated alone or in multiple instances with a singular platform (i.e., a base system 1204) to realize system 1200's full operation. The modularity allows the clinical evaluation system 1200 to be designed to leverage the same synchronously acquired biophysical signals and data set and base platform to assess for the presence of several different diseases as such disease-specific algorithms are developed, thereby reducing testing and certification time and cost.

In various embodiments, different versions of the clinical evaluation system 1200 may implement the assessment system 103 (FIG. 1) by having included containing different feature computation modules that can be configured for a given disease state(s), medical condition(s), or indicating condition(s) of interest. In another embodiment, the clinical evaluation system 1200 may include more than one assessment system 103 and may be selectively utilized to generate different scores specific to a classifier 116 of that engine 103. In this way, the modules of FIGS. 1 and 12A in a more general sense may be viewed as one configuration of a modular system in which different and/or multiple engines 103, with different and/or multiple corresponding classifiers 116, may be used depending on the configuration of module desired. As such, any number of embodiments of the modules of FIG. 1, with or without power spectral-based specific feature(s), may exist.

In FIG. 12A, System 1200 can analyze one or more biophysical-signal data sets (e.g., 110) using machine-learned disease-specific algorithms to assess for the likelihood of elevated LVEDP, as one example, of pathology or abnormal state. System 1200 includes hardware and software components that are designed to work together in combination to facilitate the analysis and presentation of an estimation score using the algorithm to allow a physician to use that score, e.g., to assess for the presence or non-presence of a disease state, medical condition, or an indication of either.

The base system 1204 can provide a foundation of functions and instructions upon which each add-on module 1202 (which includes the disease-specific algorithm) then interfaces to assess for the pathology or indicating condition. The base system 1204 as shown in the example of FIG. 12A includes a base analytical engine or analyzer 1206, a web-service data transfer API 1208 (shown as “DTAPI” 1208), a report database 1210, a web portal service module 1213, and the data repository 111 (shown as 112a).

Data repository 112a, which can be cloud-based, stores data from the signal capture system 102 (shown as 102b). Biophysical signal capture system 102b, in some embodiments, is a reusable device designed as a single unit with a seven-channel lead set and photoplethysmogram (PPG) sensor securely attached (i.e., not removable). Signal capture system 102b, together with its hardware, firmware, and software, provides a user interface to collect patient-specific metadata entered therein (e.g., name, gender, date of birth, medical record number, height, and weight, etc.) to synchronously acquire the patient's electrical and hemodynamic signals. The signal capture system 102b may securely transmit the metadata and signal data as a single data package directly to the cloud-based data repository. The data repository 112a, in some embodiments, is a secure cloud-based database configured to accept and store the patient-specific data package and allow for its retrieval by the analytical engines or analyzer 1206 or 1214.

Base analytical engine or analyzer 1206 is a secure cloud-based processing tool that may perform quality assessments of the acquired signals (performed via “SQA” module 1216), the results of which can be communicated to the user at the point of care. The base analytical engine or analyzer 1206 may also perform pre-processing (shown via pre-processing module 1218) of the acquired biophysical signals (e.g., 110—see FIG. 1). Web portal 1213 is a secure web-based portal designed to provide healthcare providers access to their patient's reports. An example output of the web portal 1213 is shown by visualization 1236. The report databases (RD) 1212 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient's general health record. In some embodiments, web portal 1213 is accessed by a call center to provide the output clinical information over a telephone. Database 1212 may be accessed by other systems that can generate a report to be delivered via the mail, courier service, personal delivery, etc.

Add-on module 1202 includes a second part 1214 (also referred to herein as the analytical engine (AE) or analyzer 1214 and shown as “AE add-on module” 1214) that operates with the base analytical engine (AE) or analyzer 1206. Analytical engine (AE) or analyzer 1214 can include the main function loop of a given disease-specific algorithm, e.g., the feature computation module 1220, the classifier model 1224 (shown as “Ensemble” module 1224), and the outlier assessment and rejection module 1224 (shown as “Outlier Detection” module 1224). In certain modular configurations, the analytical engines or analyzers (e.g., 1206 and 1214) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient's score using the disease-specific algorithm. To execute the analysis pipeline, AE add-on module 1214 can include and execute instructions for the various feature modules 114 and classifier module 116 as described in relation to FIG. 1 to determine an output score (e.g., 118) of the metrics associated with the physiological state of a patient. The analysis pipeline in the AE add-on module 1214 can compute the features or parameters (shown as “Feature Computation” 1220) and identifies whether the computed features are outliers (shown as “Outlier Detection” 1222) by providing an outlier detection return for a signal-level response of outlier vs. non-outlier based on the feature. The outliers may be assessed with respect to the training data set used to establish the classifier (of module 116). AE add-on module 1214 may generate the patient's output score (e.g., 118) (e.g., via classifier module 1224) using the computed values of the features and classifier models. In the example of an evaluation algorithm for the estimation of elevated LVEDP, the output score (e.g., 118) is an LVEDP score. For the estimation of CAD, the output score (e.g., 118) is a CAD score.

The clinical evaluation system 1200 manages the data within and across components using the web-service DTAPIs 1208 (also may be referred to as HCPP web services in some embodiments). DTAPIs 1208 may be used to retrieve acquired biophysical data sets from, and to store signal quality analysis results to, the data repository 112a. DTAPIs 1208 may also be invoked to retrieve and provide the stored biophysical data files to the analytical engines or analyzers (e.g., 1206, 1214), and the results of the analytical engine's analysis of the patient signals may be transferred using DTAPI 1208 to the report database 1210. DTAPIs 1208 may also be used, upon a request by a healthcare professional, to retrieve a given patient data set to the web portal module 1213, which may present a report to the healthcare practitioner for review and interpretation in a secure web-accessible interface.

Clinical evaluation system 1200 includes one or more feature libraries 1226 that store the power spectral-based features 120 and various other features of the feature modules 122. The feature libraries 1226 may be a part of the add-on modules 1202 (as shown in FIG. 12A) or the base system 1204 (not shown) and are accessed, in some embodiments, by the AE add-on module 1214.

Further details of the modularity of modules and various configurations are provided in U.S. Provisional Patent Application No. 63/235,960, filed Aug. 19, 2021, entitled “Modular Disease Assessment System,” which is hereby incorporated by reference herein in its entirety.

Example Operation of the Modular Clinical Evaluation System

FIG. 12B shows a schematic diagram of the operation and workflow of the analytical engines or analyzers (e.g., 1206 and 1214) of the clinical evaluation system 1200 of FIG. 12A in accordance with an illustrative embodiment.

Signal quality assessment/rejection (1230). Referring to FIG. 12B, the base analytical engine or analyzer 1206 assesses (1230), via SQA module 1216, the quality of the acquired biophysical-signal data set while the analysis pipeline is executing. The results of the assessment (e.g., pass/fail) are immediately returned to the signal capture system's user interface for reading by the user. Acquired signal data that meet the signal quality requirements are deemed acceptable (i.e., “pass”) and further processed and subjected to analysis for the presence of metrics associated with the pathology or indicating condition (e.g., elevated LVEDP or mPAP, CAD, PH/PAH, abnormal LVEF, HFpEF) by the AE add-on module 1214. Acquired signals deemed unacceptable are rejected (e.g., “fail”), and a notification is immediately sent to the user to inform the user to immediately obtain additional signals from the patient (see FIG. 2).

The base analytical engine or analyzer 1206 performs two sets of assessments for signal quality, one for the electrical signals and one for the hemodynamic signals. The electrical signal assessment (1230) confirms that the electrical signals are of sufficient length, that there is a lack of high-frequency noise (e.g., above 170 Hz), and that there is no power line noise from the environment. The hemodynamic signal assessment (1230) confirms that the percentage of outliers in the hemodynamic data set is below a pre-defined threshold and that the percentage and maximum duration that the signals of the hemodynamic data set are railed or saturated is below a pre-defined threshold.

Feature Value Computation (1232). The AE add-on module 1214 performs feature extraction and computation to calculate feature output values. In the example of the LVEDP algorithm, the AE add-on module 1214 determines, in some embodiments, a total of 446 feature outputs belonging to 18 different feature families (e.g., generated in modules 120 and 122), including the power spectral-based features (e.g., generated in module 120). For the CAD algorithm, an example implementation of the AE add-on module 1214 determines a set of features, including 456 features corresponding to the same 18 feature families.

Additional descriptions of the various features, including those used in the LVEDP algorithm and other features and their feature families, are described in U.S. Provisional Patent Application No. 63/235,960, filed Aug. 23, 2021, entitled “Method and System to Non-Invasively Assess Elevated Left Ventricular End-Diastolic Pressure”; U.S. Provisional Patent Application No. 63/236,072, filed Aug. 23, 2021, entitled “Methods and Systems for Engineering Visual Features From Biophysical Signals for Use in Characterizing Physiological Systems”; U.S. Provisional Patent Application No. 63/235,966, filed Aug. 23, 2021, entitled “Method and System for Engineering Rate-Related Features From Biophysical Signals for Use in Characterizing Physiological Systems”; U.S. Provisional Patent Application No. 63/235,968, filed Aug. 23, 2021, entitled “Methods and Systems for Engineering Wavelet-Based Features From Biophysical Signals for Use in Characterizing Physiological Systems”; U.S. Provisional Patent Application No. 63/130,324, titled “Method and System to Assess Disease Using Cycle Variability Analysis of Cardiac and Photoplethysmographic Signals”; U.S. Provisional Patent Application No. 63/235,971, filed Aug. 23, 2021, entitled “Methods and Systems for Engineering photoplethysmographic Waveform Features for Use in Characterizing Physiological Systems”; U.S. Provisional Patent Application No. 63/236,193, filed Aug. 23, 2021, entitled “Methods and Systems for Engineering Cardiac Waveform Features From Biophysical Signals for Use in Characterizing Physiological Systems” having attorney docket no. 10321-055pv1; U.S. Provisional Patent Application No. 63/235,974, filed Aug. 23, 2021, entitled “Methods and Systems for Engineering Conduction Deviation Features From Biophysical Signals for Use in Characterizing Physiological Systems,” each of which is hereby incorporated by reference herein in its entirety.

Classifier Output Computation (1234). The AE add-on module 1214 then uses the calculated feature outputs in classifier models (e.g., machine-learned classifier models) to generate a set of model scores. The AE add-on module 1214 joins the set of model scores in an ensemble of the constituent models, which, in some embodiments, averages the outputs of the classifier models as shown in Equation 14 in the example of the LVEDP algorithm.

$$Ensemble estimation = \frac{{Model}_{1} + {Model}_{2} + … + {Model}_{n}}{n} \qquad ( Equation 14 )$$

In some embodiments, classifier models may include models that are developed based on ML techniques described in U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

In the example of the LVEDP algorithm, thirteen (13) machine-learned classifier models are each calculated using the calculated feature outputs. The 13 classifier models include four ElasticNet machine-learned classifier models, four RandomForestClassifier machine-learned classifier models, and five extreme gradient boosting (XGB) classifier models. In some embodiments, the patient's metadata information, such as age, gender, and BMI value, may be used. The output of the ensemble estimation may be a continuous score. The score may be shifted to a threshold value of zero by subtracting the threshold value for presentation within the web portal. The threshold value may be selected as a trade-off between sensitivity and specificity. The threshold may be defined within the algorithm and used as the determination point for test positive (e.g., “Likely Elevated LVEDP”) and test negative (e.g., “Not Likely Elevated LVEDP”) conditions.

In some embodiments, the analytical engine or analyzer can fuse the set of model scores with a body mass index-based adjustment or an adjustment based on age or gender. For example, the analytical engine or analyzer can average the model estimation with a sigmoid function of the patient BMI having the form

$$sigmoid ( x ) = \frac{1}{1 + {e}^{- x}} .$$

Physician Portal Visualization (1236). The patient's report may include a visualization 1236 of the acquired patient data and signals and the results of the disease analyses. The analyses are presented, in some embodiments, in multiple views in the report. In the example shown in FIG. 12B, the visualization 1236 includes a score summary section 1240 (shown as “Patient LVEDP Score Summary” section 1240), a threshold section 1242 (shown as “LVEDP Threshold Statistics” section 1242), and a frequency distribution section 1244 (shown as “Frequency Distribution” section 1208). A healthcare provider, e.g., a physician, can review the report and interpret it to provide a diagnosis of the disease or to generate a treatment plan.

The healthcare portal may list a report for a patient if a given patient's acquired signal data set meets the signal quality standard. The report may indicate a disease-specific result (e.g., elevated LVEDP) being available if the signal analysis could be performed. The patient's estimated score (shown via visual elements 118a, 118b, 118c) for the disease-specific analysis may be interpreted relative to an established threshold.

In the score summary section 1240 shown in the example of FIG. 12B, the patient's score 118a and associated threshold are superimposed on a two-tone color bar (e.g., shown in section 1240) with the threshold located at the center of the bar with a defined value of “0” representing the delineation between test positive and test negative. The left of the threshold may be lightly shaded light and indicates a negative test result (e.g., “Not Likely Elevated LVEDP”) while to the right of the threshold may be darkly shaded to indicate a positive test result (e.g., “Likely Elevated LVEDP”).

The threshold section 1242 shows reported statistics of the threshold as provided to a validation population that defines the sensitivity and specificity for the estimation of the patient score (e.g., 118). The threshold is the same for every test regardless of the individual patient's score (e.g., 118), meaning that every score, positive or negative, may be interpreted for accuracy in view of the provided sensitivity and specificity information. The score may change for a given disease-specific analysis as well with the updating of the clinical evaluation.

The frequency distribution section 1244 illustrates the distribution of all patients in two validation populations (e.g., (i) a non-elevated population to indicate the likelihood of a false positive estimation and (ii) an elevated population to indicate a likelihood of a false negative estimation). The graphs (1246, 1248) are presented as smooth histograms to provide context for interpreting the patient's score 118 (e.g., 118b, 118c) relative to the test performance validation population patients.

The frequency distribution section 1240 includes a first graph 1246 (shown as “Non-Elevated LVEDP Population” 1246) that shows the score (118b), indicating the likelihood of the non-presence of the disease, condition, or indication, within a distribution of a validation population having non-presence of that disease, condition, or indication and a second graph 1248 (shown as “Elevated LVEDP Population” 1248) that shows the score (118c), indicates the likelihood of the presence of the disease, condition, or indication, within a distribution of validation population having the presence of that disease, condition, or indication. In the example of the assessment of elevated LVDEP, the first graph 1246 shows a non-elevated LVEDP distribution of the validation population that identifies the true negative (TN) and false positive (FP) areas. The second graph 1248 shows an elevated LVEDP distribution of the validation population that identifies the false negative (TN) and true positive (FP) areas.

The frequency distribution section 1240 also includes the interpretative text of the patient's score relative to other patients in a validation population group (as a percentage). In this example, the patient has an LVEDP score of −0.08, which is located to the left side of the LVEDP threshold, indicating that the patient has “Not Likely Elevated LVEDP.”

The report may be presented in the healthcare portal, e.g., to be used by a physician or healthcare provider in their diagnosis for indications of left-heart failure. The indications include, in some embodiments, a probability or a severity score for the presence of a disease, medical condition, or an indication of either.

Outlier Assessment and Rejection Detection (1238). Following the AE add-on module 1214 computing the feature value outputs (in process 1232) and prior to their application to the classifier models (in process 1234), the AE add-on module 1214 is configured in some embodiments to perform outlier analysis (shown in process 1238) of the feature value outputs. Outlier analysis evaluation process 1238 executes a machine-learned outlier detection module (ODM), in some embodiments, to identify and exclude anomalous acquired biophysical signals by identifying and excluding anomalous feature output values in reference to the feature values generated from the validation and training data. The outlier detection module assesses for outliers that present themselves within sparse clusters at isolated regions that are out of distribution from the rest of the observations. Process 1238 can reduce the risk that outlier signals are inappropriately applied to the classifier models and produce inaccurate evaluations to be viewed by the patient or healthcare provider. The accuracy of the outlier module has been verified using hold-out validation sets in which the ODM is able to identify all the labeled outliers in a test set with the acceptable outlier detection rate (ODR) generalization.

While the methods and systems have been described in connection with certain embodiments and specific examples, it is not intended that the scope be limited to the particular embodiments set forth, as the embodiments herein are intended in all respects to be illustrative rather than restrictive. The power spectral-based features discussed herein may ultimately be employed to make, or to assist a physician or other healthcare provider in making, noninvasive diagnoses or determinations of the presence or non-presence and/or severity of other diseases, medical conditions, or indication of either, such as, e.g., coronary artery disease, pulmonary hypertension and other pathologies as described herein using similar or other development approaches. In addition, the example analysis, including the spectral-based features, can be used in the diagnosis and treatment of other cardiac-related pathologies and indicating conditions as well as neurological-related pathologies and indicating conditions, such assessment can be applied to the diagnosis and treatment (including, surgical, minimally invasive, and/or pharmacologic treatment) of any pathologies or indicating conditions in which a biophysical signal is involved in any relevant system of a living body. One example in the cardiac context is the diagnosis of CAD, and other diseases, medical condition, or indicating conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or indicating conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, indicating conditions and/or diseases. Non-limiting examples of neurological-related diseases, pathologies or indicating conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, indicating conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/indicating conditions and vision-related diseases/indicating conditions.

In addition, the clinical evaluation system described herein may be configured to analyze biophysical signals such as an electrocardiogram (ECG), electroencephalogram (EEG), gamma synchrony, respiratory function signals, pulse oximetry signals, perfusion data signals; quasi-periodic biological signals, fetal ECG signals, blood pressure signals; cardiac magnetic field signals, heart rate signals, among others.

Further examples of processing that may be used with the exemplified method and system disclosed herein are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. patent application Ser. Nos. 16/831,264; 16/831,380; 17/132,869; PCT Application nos. PCT/IB2020/052889; PCT/IB2020/052890, each of which is hereby incorporated by reference herein in its entirety.

The following patents, applications, and publications as listed below and throughout this document are hereby incorporated by reference in their entirety herein.

### LIST OF REFERENCES

[1] H. Abbaspour, S. M. Razavi, and N. Mehrshad, “Electrocardiogram based identification using a new effective intelligent selection of fused features,” j. med. signals sensors, vol. 5, no. 1, pp. p 30-39, 2015.
[2] C. H. Lin, “Frequency-domain features for ECG beat discrimination using grey relational analysis-based classifier,” comput. math. with appl., vol. 55, no. 4, pp. 680-690, 2008.
[3] H. Gothwal, S. Kedawat, and R. Kumar, “Cardiac arrhythmias detection in an ECG beat signal using fast Fourier transform and artificial neural network,” j. biomed. sci. eng., vol. 4, pp. 289-296, 2011.
[4] C. Maier, H. Dickhaus, and J. Gittinger, “Unsupervised morphological classification of QRS complexes,” in computers in cardiology. vol. 26,” pp. 683-686, 1999.
[5] M. F. Shinwari, N. Ahmed, H. Humayun, I. Haq, S. Haider, and A. Anam, “Classification algorithm for feature extraction using linear discriminant analysis and cross-correlation on ECG signals,” int. j. adv. sci. technol., vol. 48, pp. 149-162, 2012.
[6] A. Maheshwari et al., “Low heart rate variability in a 2-minute electrocardiogram recording is associated with an increased risk of sudden cardiac death in the general population: the atherosclerosis risk in communities study,” 2016.
[7] A. K. Bhoi, K. S. Sherpa, And B. Khandelwal, “Ischemia and arrhythmia classification using time-frequency domain features of QRS complex,” procedia computer science, vol. 132, no. 4, pp. 606-613, 2018.
[8] J. D. Blood et al., “The variable heart: high frequency and very low frequency correlates of depressive symptoms in children and adolescents” j affect disord, vol. 186, pp. 119-126, 2015.
[9] chandan K Karmakar, A. H. Khandoker, and M. Palaniswami, “power spectral analysis of ecg signals during obstructive sleep apnoea hypopnoea epochs,” in proceedings of the 2007 international conference on intelligent sensors, pp. 573-576, 2007.
[10] H. M. Madsen, “spectral decomposition of electrocardiograms for the diagnosis of pulmonary hypertension and the estimation of invasively measured parameters,” M.S. thesis, College of BioEng., Univ of Colorado, Colorado, 2017.
[11] J. W. Cooley and J. W. Tukey, “An algorithm for the machine calculation of complex Fourier series,” math. comput., vol. 19, no. 90, pp. 297-301, 1965.
[12] J. Harris and F. J. Harris, “On the use of windows for harmonic analysis with the discrete Fourier transform,” proc. ieee, vol. 66, no. 1, pp. 51-83, 1978.
[13] S. Das and M. Chakraborty, “Comparison of power spectral density (PSD) of normal and abnormal ECGs,” int. j. comput. appl.—ijca, vol. 2, no. 3, pp. 10-14, 2011.
[14] L. G. Tereshchenko and M. E. Josephson, “Frequency content and characteristics of ventricular conduction,” journal of electrocardiology, vol. 48, no. 6, pp. 933-937, 2015.
[15] R. W. Makuch, D. H. Freeman, and M. F. Johnson, “Justification for the lognormal distribution as a model for blood pressure,” j. chronic dis., vol. 32, no. 3, pp. 245-250, 1979.
[16] K. Mizuseki and G. Buzsiki, “Preconfigured, skewed distribution of firing rates in the hippocampus and entorhinal cortex,” cell rep., vol. 4, no. 5, pp. 1010-1021, 2013.
[17] Rosenmann, David, Yaakov Mogilevski, Guy Amit, Linda R. Davrath, and Dan Tzivoni. 2013. “High-Frequency QRS Analysis Improves the Specificity of Exercise ECG Testing in Women Referred for Angiography.” Journal of Electrocardiology 46 (1): 19-26. https://doi.org/10.1016/j.jelectrocard.2012.08.007.
[18] Tragirdh, Elin, Olle Pahlm, Galen S. Wagner, and Jonas Pettersson. 2004. “Reduced High-Frequency QRS Components in Patients with Ischemic Heart Disease Compared to Normal Subjects.” Journal of Electrocardiology 37 (3): 157-62. https://doi.org/10.1016/j.jelectrocard.2004.02.004.

What is claimed is:

1. A method for non-invasively assessing a disease state, abnormal condition, or an indication of either of a subject, the method comprising:
obtaining, by one or more processors, a biophysical signal data set of the subject comprising two or more biophysical signals;
determining, by the one or more processors, values of power spectral and coherence-based features or parameters that (i) characterize signal energy or power in the frequency domain through a decomposition of the two or more biophysical signals into its frequency components and/or (ii) measure an association between the frequency content of the two or more biophysical signals; and
determining, by the one or more processors, an estimated value for a presence of the disease state, abnormal condition, or indication of either based, in part, on the determined values of the power spectral and coherence-based features or parameters, wherein the estimated value for the of the disease state, abnormal condition, or indication of either is used in a model to non-invasively estimate the presence of an expected disease state, abnormal condition, or indication of either,
wherein the estimated value is subsequently outputted for use in a diagnosis of the expected disease state, abnormal condition, or indication of either or to direct treatment of the expected disease state or condition.

2. The method of claim 1, wherein the biophysical signal data set comprises biopotential signals acquired for three channels of measurements.

3. The method of claim 1, wherein the biophysical signal data set comprises photoplethysmographic signals acquired from optical sensors.

4. The method of claim 1, wherein the biophysical signal data set comprises (i) biopotential signals acquired for three channels of measurements and (ii) photoplethysmographic signals acquired from optical sensors.

5. The method of claim 1, wherein the step of determining (i) the values of the one or more power spectral density associated properties or (ii) the coherence of two or more power spectral density associated properties comprises:
generating, by the one or more processors, a power spectral density model of the biophysical signal data set, wherein the power spectral density model comprises a power of a signal of the biophysical signal data set;
determining, by the one or more processors, one or more values of features extracted from the power spectral density model, wherein the one or more features include at least one of:
a feature associated with a decay function fitted to peaks defined in a low-frequency portion of the power spectral density model;
a feature associated with a decay function fitted to peaks defined in a high-frequency portion of the power spectral density model;
a feature associated with a linear function fitted to peaks defined in the low-frequency portion of the power spectral density model;
a feature associated with a linear function fitted to peaks defined in the high-frequency portion of the power spectral density model;
a feature associated with a power function applied to the low-frequency portion of the power spectral density model;
a feature associated with a power function applied to the high-frequency portion of the power spectral density model; or
a feature associated with a transition frequency determined between the low-frequency portion and the high-frequency portion of the power spectral density model.

6. The method of claim 1, wherein the step of determining (i) the values of the one or more power spectral density associated properties or (ii) the coherence of two or more power spectral density associated properties comprises:
generating, by the one or more processors, a power spectral density model of the biophysical signal data set, wherein the power spectral density model comprises a power of a signal of the biophysical signal data set;
determining, by the one or more processors, one or more values of features extracted from the power spectral density model, wherein the one or more features are selected from the group consisting of:
a feature associated with a ratio of (i) a power function applied to the low-frequency portion of the power spectral density model to (ii) a power function applied to the high-frequency portion of the power spectral density model;
a feature associated with a ratio of (i) a power function applied to the low-frequency portion of the power spectral density model of a first signal of the biophysical data set to (ii) a power function applied to the lower frequency portion of the power spectral density model of a second signal of the biophysical data set; or
a feature associated with a ratio of (i) a power function applied to fundamental frequency portions of the power spectral density model of the first signal of the biophysical data set to (ii) a power function applied to fundamental frequency portions of the power spectral density model of the second signal of the biophysical data set.

7. The method of claim 1, wherein the step of determining (i) the values of the one or more power spectral density associated properties or (ii) the coherence of two or more power spectral density associated properties comprises:
generating, by the one or more processors, a coherence spectral model using two or more power spectral density models associated with two or more signals of the biophysical signal data set; and
determining, by the one or more processors, one or more values of features extracted from the coherence spectral model, wherein the one or more features comprises a feature associated with a statistical assessment of coherence distributions determined between the first signal of the biophysical signal data set and the second signal of the biophysical signal data set.

8. The method of claim 1, wherein the step of determining (i) the values of the one or more power spectral density associated properties or (ii) the coherence of two or more power spectral density associated properties comprises:
generating, by the one or more processors, a coherence spectral model of two or more power spectral density models associated with two or more signals of the biophysical signal data set; and
determining, by the one or more processors, one or more values of features extracted from the coherence spectral model, wherein the one or more features include at least one of:
a feature associated with a combined coherence determined between the first signal of the biophysical signal data set;
a feature associated with a combined coherence determined among all signals of the biophysical signal data set;
a feature associated with a mean of coherence distributions determined between the first signal of the biophysical signal data set and the second signal of the biophysical signal data set;
a feature associated with a median of the coherence distributions determined between the first signal of the biophysical signal data set and the second signal of the biophysical signal data set;
a feature associated with a standard deviation of the coherence distributions determined between the first signal of the biophysical signal data set and the second signal of the biophysical signal data set;
a feature associated with a skewness of the coherence distributions determined between the first signal of the biophysical signal data set and the second signal of the biophysical signal data set;
a feature associated with a kurtosis of the coherence distributions determined between the first signal of the biophysical signal data set and the second signal of the biophysical signal data set;
a feature associated with an entropy of coherence distributions determined between the first signal of the biophysical signal data set and the second signal of the biophysical signal data set; or
a feature associated with a sum square of residuals between (i) a model fitted of a coherence distribution determined between the first signal of the biophysical data set and the second signal of the biophysical data set and (ii) the coherence distribution determined between the first signal of the biophysical data set and the second signal of the biophysical data set.

9. The method of claim 1, wherein the step of determining (i) the values of the one or more power spectral density associated properties or (ii) the coherence of two or more power spectral density associated properties comprises estimating respective power spectrums by (i) dividing each respective signal of the biophysical signal data set into successive blocks to form a periodogram for each block and (ii) averaging the periodogram for each block to obtain a statistical representation of the power spectrum.

10. The method of claim 1, wherein the step of determining (i) the values of the one or more power spectral density associated properties or (ii) the coherence of two or more power spectral density associated properties comprises estimating respective power spectrums using a spectral window selected from the group consisting of a Hann spectral window, a Hamming spectral window, a Blackman spectral window, a Gaussian spectral window, a Tukey spectral window, and a Welch spectral window.

11. The method of claim 1, wherein the step of determining (i) the values of the one or more power spectral density associated properties or (ii) the coherence of two or more power spectral density associated properties comprises:
generating, by the one or more processors, a power spectral density model of the biophysical signal data set, wherein the power spectral density model comprises a power of a signal of the biophysical signal data set;
determining, by the one or more processors, one or more values of features extracted from the power spectral density model, wherein the one or more features include at least one of:
a feature associated with a cumulative power in the power spectral density model of a signal in the biophysical signal data set;
a feature associated with a cumulative power in the power spectral density models of all signals in the biophysical signal data set; or
a feature associated with a ratio of (i) the cumulative power in the power spectral density model of the first signal, the second signal, or the third signal to (ii) the cumulative power in the power spectral density models of all signals in the biophysical signal data set; and

12. The method of claim 1 further comprising:
causing, by the one or more processors, generation of a visualization of the estimated value for the presence of the disease state, abnormal condition, or indication of either, wherein the generated visualization is rendered and displayed at a display of a computing device and/or presented in a report.

13. The method of claim 1, wherein the values of the one or more power spectral density associated properties or the coherence of two or more power spectral density associated properties are used in the model selected from the group consisting of a linear model, a decision tree model, a random forest model, a support vector machine model, a neural network model.

14. The method of claim 13, wherein the model further includes features selected from the group consisting of:
one or more depolarization or repolarization wave propagation associated features;
one or more depolarization wave propagation deviation associated features;
one or more cycle variability associated features;
one or more dynamical system associated features;
one or more cardiac waveform topologic and variations associated features;
one or more PPG waveform topologic and variations associated features;
one or more cardiac or PPG signal power spectral density associated features;
one or more cardiac or PPG signal visual associated features; and
one or more predictability features.

15. The method of claim 1, wherein the disease state, abnormal condition, or indication of either is selected from the group consisting of coronary artery disease, pulmonary hypertension, pulmonary arterial hypertension, pulmonary hypertension due to left heart disease, rare disorders that lead to pulmonary hypertension, left ventricular heart failure or left-sided heart failure, right ventricular heart failure or right-sided heart failure, systolic heart failure, diastolic heart failure, ischemic heart disease, and arrhythmia.

16. The method of claim 1, further comprising:
acquiring, by one or more acquisition circuits of a measurement system, voltage gradient signals over the one or more channels, wherein the voltage gradient signals are acquired at a frequency greater than about 1 kHz; and
generating, by the one or more acquisition circuits, the obtained biophysical data set from the acquired voltage gradient signals.

17. The method of claim 1, further comprising:
acquiring, by one or more acquisition circuits of a measurement system, one or more photoplethysmographic signals; and
generating, by the one or more acquisition circuits, the obtained biophysical data set from the acquired voltage gradient signals.

18. The method of claim 1, wherein the one or more processors are located in a cloud platform or a local computing device.

19. A system comprising:
a processor; and
a memory having instructions stored thereon, wherein execution of the instructions by the processor causes the processor to:
obtain a biophysical signal data set of a subject comprising two or more biophysical signals;
determine values of power spectral and coherence-based features or parameters that (i) characterize signal energy or power in the frequency domain through a decomposition of the two or more biophysical signals into its frequency components and/or (ii) measure an association between the frequency content of the two or more biophysical signals; and
determine an estimated value for a presence of the disease state, abnormal condition, or indication of either based, in part, on the determined values of the power spectral and coherence-based features or parameters, wherein the estimated value for the of the disease state, abnormal condition, or indication of either is used in a model to non-invasively estimate the presence of an expected disease state, abnormal condition, or indication of either,
wherein the estimated value is subsequently outputted for use in a diagnosis of the expected disease state, abnormal condition, or indication of either or to direct treatment of the expected disease state or condition.

20. A non-transitory computer-readable medium having instructions stored thereon, wherein execution of the instructions by a processor causes the processor to:
obtain a biophysical signal data set of a subject comprising two or more biophysical signals;
determine values of power spectral and coherence-based features or parameters that (i) characterize signal energy or power in the frequency domain through a decomposition of the two or more biophysical signals into its frequency components and/or (ii) measure an association between the frequency content of the two or more biophysical signals; and
determine an estimated value for a presence of the disease state, abnormal condition, or indication of either based, in part, on the determined values of the power spectral and coherence-based features or parameters, wherein the estimated value for the of the disease state, abnormal condition, or indication of either is used in a model to non-invasively estimate the presence of an expected disease state, abnormal condition, or indication of either,
wherein the estimated value is subsequently outputted for use in a diagnosis of the expected disease state, abnormal condition, or indication of either or to direct treatment of the expected disease state or condition.
